(19) United States
(12) Patent Application Publication
(10) Pub. No.: US 2011/0300186 A1
(43) Pub. Date: Dec. 8, 2011

(54) Functionalized Nano- and Micro-materials for Medical Therapies

(75) Inventors: Karl Erik Hellstrom, Seattle, WA (US); Ingegerd Hellstrom, Seattle, WA (US); Pu Liu, Seattle, WA (US); Huafeng Wei, Seattle, WA (US); Jun Liu, Richland, WA (US); Chenghong Lei, Richland, WA (US); Baowei Chen, Richland, WA (US); Xiaolin Li, Richland, WA (US)

(73) Assignees: Battelle Memorial Institute, Richland, WA (US); University of Washington through its Center for Commercialization, Seattle, WA (US)

(21) Appl. No.: 13/087,164

(22) Filed: Apr. 14, 2011

Related U.S. Application Data
(60) Provisional application No. 61/323,966, filed on Apr. 14, 2010.

(51) Int. Cl. A61K 9/00; A61K 31/7088; A61P 35/00; A61K 39/00; A61P 37/04; A61K 38/02; A61K 39/395

(52) U.S. Cl. 424/400; 514/19.3; 514/44 R; 424/172.1; 424/277.1; 424/178.1

(57) ABSTRACT

Compositions containing an optionally surface-functionalized mesoporous support and a biologically active agent, and pharmaceutical compositions of the same, are provided herein. Such compositions can be useful in the treatment of tumors, for example, by injection of the composition at a location near the site of the tumor.

### CROSS-REFERENCE TO RELATED APPLICATION

This application claims the benefit of the filing date of U.S. Provisional Application No. 61/323,966, filed Apr. 14, 2010, which is hereby incorporated by reference in its entirety.

### STATEMENT OF GOVERNMENT SUPPORT

The invention described herein was made in part with government support under grant numbers R01GM080987 and R01CA134487, each awarded by the National Institutes of Health; as well as funds provided under Contract DE-AC0576RL01830 awarded by the U.S. Department of Energy. The Government has certain rights in the invention.

### FIELD OF THE INVENTION

The present disclosure relates to compositions for treating tumors, and methods for their use by local administration near the site of the tumor.

### BACKGROUND OF THE INVENTION

A fundamental issue in cancer therapy is that cancer' cells undergo extensive DNA changes and that their genes mutate at a very high rate, leading to variants which are resistant to the original therapy, including cytotoxic drugs. While the mutations can provide novel epitopes for recognition by the immune system, the high mutability of tumor cell populations is a problem for immunotherapy that targets one or a couple of tumor antigens due to the frequent occurrence of variants that have lost a given tumor antigen or the ability to present it via MHC. This problem may be overcome by strategies that are capable of generating and expanding a strong immune response at the tumor site, including tumor-draining lymph nodes, which is directed to a large number of tumor specific and tumor-selective epitopes and is capable of destroying tumor cells both at the original and distant (untreated) sites. Systemic administration of immunologically active (IA) biomolecules has rapidly developed into a large pharmaceutical industry.

The tumor micro-environment is highly immunosuppressive because of its high concentration of tumor antigen, regulatory T lymphocytes, TGFβ and IDO, etc. It is therefore important that a sufficient amount of IA biomolecules get delivered to the tumor to decrease immunosuppression. To accomplish this by systemic administration, large doses and short dose intervals are needed which increases the risk for serious side effects, such as autoimmunity-based colitis and pituitary damage in patients receiving a monoclonal antibody to the immunoregulatory molecule CTLA4, by inducing autoimmunity to normal tissue antigens.

Another major problem with current systemic delivery has been resistance of the tissues to the influx of the biologically active molecules. Direct injection of tumors, is also problematic, in that there is resistance of the tissues to the influx of the biologically active molecules within heterogenius tissue, backflow and diversion through the point of entry. This results in low quantities remaining in the tumor tissue to be treated. Methods which could provide increased penetration and/or reduced backflow and diversion through the point of entry, so that more material is introduced into and remains in the tumor, would offer considerable therapeutic advantage.

Therefore, there is a need for a technology that provides a sustainable local delivery to tumors of agents which can counteract the immunosuppressive mechanisms at the tumor site to induce a systemic immune response against the many antigens expressed by the given tumor capable of destroying both the local tumor and untreated distant metastases.

### SUMMARY OF THE INVENTION

In one aspect, the invention provides compositions comprising (i) a mesoporous support having an optional surface functionalization, wherein the surface functionalization, when present, comprises functional groups capable of associating with a biologically active agent; and (ii) a biologically active agent, wherein at least a portion of the biologically active agent is contained within the pores of the mesoporous support.

In another aspect, the invention provides pharmaceutical compositions comprising the composition of preceding aspect and a pharmaceutically acceptable carrier.

In another aspect, the invention provides methods for treating a tumor comprising inserting at a site near a tumor in a patient in need of treatment a therapeutically effective amount of a composition comprising (i) a mesoporous support having an optional surface functionalization, wherein the surface functionalization, when present, comprises functional groups capable of associating with the biologically active agent; and (ii) a biologically active agent, wherein at least a portion of the biologically active agent is contained within the pores of the mesoporous support.

By providing an prolonged or controlled release of tumor antigen, antibody, or antibody-conjugate, and immunoregulatory signals locally in tumors and at vaccination sites, mesoporous supports entrapping one or more biologically active agents (e.g., immunologically active proteins including antibodies) can induce a more effective tumor-destructive immune response with less side effects, an at lower dosage levels than currently available immunotherapeutic techniques for cancers.

Since biologically active agents can be slowly released from the mesoporous support particles over a prolonged time period, delivery via such particles does not cause the high peak concentration that result from injection of the same molecules that have not been entrapped in mesoporous support particles. Such slow and localized releases have been shown to generate lower toxicities as shown by the survival data herein which increases the available therapeutic window. In certain examples, a disproportionate increase in efficacy has been observed data, such that a greater response has been elicited using surprisingly lower physiological concentrations. In another advantage, injecting a tumor with, for example, an antibody via the compositions described herein, regression in distal (non-injected) tumors has been observed as described herein.

Further, the retention of the therapeutic agent in the tumor tissue, via the compositions of described herein, allows for longer contact of the diseased tissue with the therapeutic agent at higher and localized concentration. Because the therapeutic agents can be cytotoxic, or stimulate a cytotoxic response, the slow release does not adversely affect the patient to the point of limiting use of the therapy. Finally, the leakage of therapeutic agents (i.e., the biologically active agents, herein) from tumors is well documented. The methods described herein provide for retaining such agents at the tumor site that may have otherwise leaked more rapidly from the target tissue. Although, as some of the agent leaks from the tumor site into the blood stream, such agent can contribute or replenish systemic concentrations, thereby acting as a depot.

The advantage of delivering molecules directly to a tumor to induce a tumor-destructive immune response within the tumor and its draining lymph nodes is that it makes possible the generation and expansion of an immune response to the many antigens that are expressed by a given tumor, including both antigens shared by other tumors of the same and different histological types but also antigens that are unique to the given tumor, e.g. as a result of mutations and translocations. The immune response generated within the tumor has a systemic component in the form of ‘concomitant tumor immunity’, i.e. an individual with a growing tumor has a systemic immune response that can destroy distant tumors Evidence for such systemic anti-tumor immunity was observed upon treatment of tumors with a composition as described herein, yielding inhibition also of tumors that were not treated directly by injection by the composition (e.g., by using anti-CTLA4 antibody loaded functionalized mesoporous silica).

In particular, induction of an immune response within a growing tumor (and/or the tumor-draining lymph nodes) by local administration of a composition as described herein, can be used to generate and expand a systemic anti-tumor response. Such can additionally cause inhibition of an untreated tumor as shown herein (Example 4).

The compositions and methods herein particularly enable the effective treatment of advanced ovarian cancers that are localized in the peritoneal cavity (abdominal cavity) as well as other contained tumors. It opens the possibility of maintenance therapy and adjunct therapy to surgical options.

### BRIEF DESCRIPTION OF THE DRAWINGS

FIG. 1A is a TEM image of 30 nm mesoporous silica.

FIG. 1B is a TEM image of 30 nm 20% HOOC-FMS mesoporous silica.

FIG. 1C shows rat IgG loading density in FMS and gradual release of the IgG from FMS. The rat IgGs were loaded to saturation in 1.0 mg of FMS in pH 7.4, PBS. Then, the FMS-IgGs were obtained by centrifuge and removing the supernatant (the elution number: 0). Then, 250 μL of the fresh simulated body fluid buffer was used for each subsequent elution by incubating and shaking FMS-IgG in the elution buffer for 5 minutes;

FIG. 1D is a fluorescence spectra of the free rat IgG, the FMS-IgG (20% HOOCFMS, PLD=0.32 mg/mg of FMS), and the released IgG from 20% HOOC-FMS. [IgG]: 0.03 mg/mL in pH 7.4, PBS. The excitation was at 278 nm.

FIG. 2A shows the distribution of FITC labeled-rat IgG in tumor and sera after injecting 0.1 mg Rat IgG-FITC free in pH 7.4, PBS or entrapped in 20% HOOC-FMS subcutaneously on one side of the mouse back. The blank pH 7.4, PBS and 20% HOOC-FMS were used as the control samples. Controls were the PBS buffer, anti-CTLA4, the corresponding FMS, and FMS-Rat IgG.

FIG. 2B shows testing results of anti-tumor activity of FMS-anti-CTLA4 injected s.c. into small established, growing mouse melanomas (3 mice/group). 1.8 mg of FMS containing 0.5 mg Anti-CTLA4 was used.

FIG. 2C shows results of anti-tumor activity of 20% HOOC-FMS-anti-CTLA4 from a repeat experiment for the preliminary test with five mice/group which had small SW1 tumors on both sides of the back, providing ten tumor sites/group. Two tumors were completely regressed. *p<0.05.

FIG. 2D shows the survival of mice in the experiment of FIG. 2C (five mice/group).

FIG. 3 shows mouse IgG loading density in FMS and gradual release of IgG from FMSs. The mouse IgGs were loaded to saturation in 1.0 mg of FMS in pH 7.4, PBS. Then, the FMS-IgGs were obtained by centrifuge and removing the supernatant (the elution number: 0). Then, 250 μL of the fresh pH 7.4, PBS was used for each subsequent elution by incubating and shaking FMS-IgG in the elution buffer for 10 minutes.

FIG. 4 shows the concentration of IgG-FITC in the tumor supernatant (A) and the serum (B) after 0.1 mg IgG-FITC and FMS entrapped with the same amount of IgG-FITC were injected intratumorally under the same conditions.

FIG. 5 shows regression also of untreated tumors in mice similar to those in FIG. 2C but carrying two established SW1 melanomas, one of which was treated by injection of FMS particles containing anti-CTLA4 Mab while the other tumor was left untreated.

FIG. 6 shows anti-tumor activity on established SW1 melanoma of anti-CD3+ anti-CD28 monoclonal antibody entrapped in FMS particles but not of anti-CD3+ anti-CD28 antibody.

FIG. 7 shows an experiment similar to that in FIG. 6 but with a double antibody dose (1200 μg/mouse) where one mouse in the ‘free’ antibody group died from toxicity 4 days after onset of treatment.

### DETAILED DESCRIPTION OF THE INVENTION

Recent advances with functionalized nanoporous supports provide an innovative approach for entrapping proteins and for their subsequent controlled release and delivery. In a non-limiting example, proteins can be entrapped in functionalized mesoporous silica (FMS) with rigid, uniform, open nanopore geometry of tens of nanometers. Mesoporous silicas have a surface area of up to 1000 m² g⁻¹ with ordered pore surface accounting for >95%. FMS with high affinity for a protein can provide a confined and interactive nanoenvironment that increases protein activity and allow large amounts of protein loading compared to unfunctionalized mesoporous silica (UMS) or normal porous silica with the same pore size.

Accordingly, in one aspect, the present disclosure provides compositions comprising (i) a mesoporous support having an optional surface functionalization, wherein the surface functionalization, when present, comprises functional groups capable of associating with a biologically active agent; and (ii) at least one biologically active agent, wherein at least a portion of each biologically active agent is contained within the pores of the mesoporous support. The term “associating with” as used herein means that no covalent bond is formed between the biologically active entity and the support, the attraction being generally due to van der Waals forces, hydrophobic, hydrophilic, hydrogen bonding, or electrostatic attraction.

In one embodiment, the composition comprises (i) a mesoporous support having a surface functionalization, wherein the surface functionalization comprises functional groups capable of associating with a biologically active agent; and (ii) at least one biologically active agent, wherein at least a portion of each biologically active agent is contained within the pores of the mesoporous support

In another embodiment, the composition comprises (i) a mesoporous support having an optional surface functionalization, wherein the surface functionalization, when present, comprises functional groups capable of associating with a biologically active agent; and (ii) a biologically active agent, wherein at least a portion of the biologically active agent is contained within the pores of the mesoporous support

In another embodiment, the composition comprises (i) a mesoporous support having a surface functionalization, wherein the surface functionalization comprises functional groups capable of associating with a biologically active agent; and (ii) a biologically active agent, wherein at least a portion of the biologically active agent is contained within the pores of the mesoporous support

The compositions described herein further comprise one or more biologically active agent. At least a portion of each agent is present within the pores of the mesoporous support. In certain embodiments, substantially all of the one or more biologically active agent are contained within the pores of the support. In certain embodiments, substantially all the biologically active agent in the composition is contained within the pores of the mesoporous support.

The term “biologically active agent” as used herein refers to any synthetic or natural compound or protein which when introduced into the body causes a desired biological response, including, but not limited to, nucleic acids (e.g., single- or double-stranded DNA, cDNA, RNA, and PNA), antibodies (including antibody fragments, antibody conjugates), proteins (e.g., cytokines, enzymes, polypeptides, peptides), pharmaceuticals (such as vitamins, antibiotics, hormones, amino acids, metabolites and drugs), and other biomolecules (such as ligands, receptors, viral vectors, viruses, phage or even entire cells) or fragments of these compounds, and the like, and any combinations thereof. In certain embodiments, the biologically active agent is a cancer therapeutic listed in the DataMonitor Report entitled “Pipeline Insight: Molecular Targeted Cancer Therapies,” reference code no. DMHC2452, published November 2008, which is hereby incorporated by reference.

As used herein, the term “antibody” includes, but is not limited to, polyclonal antibodies, monoclonal antibodies (mAb), human, humanized or chimeric antibodies (e.g., comprising an immunoglobulin binding domain, or equivalent, fused to another polypeptide), and biologically functional antibody fragments sufficient for binding of the antibody fragment to the antigen of interest, such as single-chain variable fragment (scFv) fusion proteins, whether natural or partly or wholly synthetically produced, and derivatives thereof. For example, “antibody” as used herein refers to (a) immunoglobulin isotype polypeptides and immunologically active portions of immunoglobulin polypeptides (i.e., polypeptides of the immunoglobulin family, or fragments thereof which comprise an antigen binding domain such as Fab, scFv, Fv, dAb, Fd; and diabodies, that immunospecifically binds to a specific antigen (e.g., CD40)); examples includehuman classes IgG, IgA, IgM, IgD and IgE, or any subclass e.g. IgG1, IgG2, IgG3 and IgG4; or (b) conservatively substituted derivatives of such immunoglobulin polypeptides or fragments that immunospecifically bind to the antigen (e.g., CD40).

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin.

The term “antibody fragment” as used herein refers to a fragment of an antibody or a polypeptide that is a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having death receptor, e.g. FAS neutralization and/or binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

For example, biologically active agents can be lymphokines (e.g. IL-12), superantigens, surrogate antigens (e.g. foreign MHC antigens), and small molecules that can have too strong biological activity to give them systemically (e.g. anti-cancer drugs, including cyclophosphamide and taxol).

In certain embodiments, the biologically active agent comprises a pharmaceutical. Examples of suitable pharmaceuticals include, but are not limited to,

(1) DNA-damaging chemotherapeutic agents including, without limitation, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane);

(2) Other cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon a, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, carminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

In certain embodiments, the biologically active agent comprises a protein. The term “protein” as used herein refers to organic compounds made of amino acids arranged in a linear chain and folded into a globular or fibrous form (i.e., a stable conformation), having, for example at least 3, or 5, or 10, or 20 amino acid residues. The amino acids in a polymer are joined together by the peptide bonds between the carboxyl and amino groups of adjacent amino acid residues. The sequence of amino acids in a protein can be defined, for example, by the sequence of a gene, which is encoded in the genetic code. In general, the genetic code specifies 20 standard amino acids; however, proteins may contain other amino acids such as selenocysteine and pyrrolysine. The residues in a protein are may be chemically modified by post-translational modification, which can alter the physical and chemical properties, folding, stability, activity, and ultimately, the function of a protein. Proteins include, for example, peptides (e.g., having 3-10 or 3-20 amino acid residues), cytokines, and enzymes.

Further examples of biologically active agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ, hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; and anti-angiogenesis factors.

In certain embodiments, the biologically active agent comprises an antibody, an antibody fragment, or an antibody conjugate. In certain embodiments, the biologically active agent comprises an antibody. In certain embodiments, the biologically active agent comprises an antibody fragment. In certain embodiments, the biologically active agent comprises an antibody conjugate.

Antibody-conjugates include, but are not limited to, (1) antibodies conjugated to radiolabels and/or cytotoxic agents, such as ¹⁸F, ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb, ^{81m}Kr, ^{87m}Sr, ⁹⁰Y, ⁹⁷Ru, ^{99m}Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi, ²¹³Bi, and ²²⁵Ac; such can be coordinated via a chelating moiety, include, for example MAG 3 (mercaptoacetyltriglycine) or bispicolylamine (SAAC); derivatives of 1,4,7,10-tetraazacyclododecanetetraacetic acid (DOTA), ethylenediaminetetraacetic acid (EDTA), diethylenetriaminepentaacetic acid (DTPA) and 1-p-Isoth iocyanato-benzyl-methyl-diethylenetriaminepentaacetic acid (ITC-MX); (2) antibodies conjugated to interleukins, such as IL-1, IL-12, IL-15, and IL-18; (3) antibodies conjugated to therapeutic drugs, such as, but not limited to, calicheamicin, DM4, auristatin, doxorubicin, taxol, cyclophosphamide, carboplatin, cisplatin, or any of the pharmaceuticals noted above.

In certain embodiments, the biologically active agent is anti-CTLA4, IgG, anti-GITR, anti-TGFα, anti-TGFβ, anti-CD137, anti-CD40, anti-CD83, anti-CD28, IL-12, IL-18, anti-PD-1, anti-4-1BB, anti-OX-40, anti- IL-2, CD33, CD 52, VEGF, TNF, TNFa, VEGF, CD20, HER2, amyloid β, EGFR, RANKL, F protein of RSV, integrin α-4/β-1, Immunoglobulin E, IL-6, C5a, IL-12, CD11α, Integrin α-V/β-3, IL-5, immunoglobulin epsilon Fc receptor II, Cytotoxic T-lymphocyte protein-4, CD80, CD95, CD-55,CD19, IL-2, IL-1, _{R} CD33, carbonic anhydrase regulator, CD22, anti-EpCAM x anti-CD3, CD3, Hsp90, mucin 16, EpCam, CD3, CD4, CD30, CCR2, CD29, CD95, IL-17, IL-18, GDF-8, CSF-1, OX40 ligand, Cadherin-3, Alk-1, or Interferon aligand.

In certain embodiments, the biologically active agent is anti-CTLA4, IgG, anti-GITR, anti-TGFα, anti-TGFβ, anti-CD137, anti-CD40, anti-CD83, anti-CD28, IL-12, or IL-18

In certain embodiments, the biologically active agent comprises a nucleic acid, e.g. a cDNA specific for the E6 or E7 epitopes of HPV 16 or 18.

In certain embodiments, the biologically active agent comprises a vaccine, e.g. tyrosinase, MAGE or gp100 for vaccination against melanoma, given alone or together with a cytokine such as GMCSF or IL12, also including a vaccine in the form of FMS particles containing one or several antigens expressed by the given tumor together with immunostimulatory or immunomodulatory proteins, such as anti-CTLA 4 antibody, IL12, a combination of anti-CD3 plus anti-CD28 antibody etc.

In certain embodiments, the biologically active agent comprises a cytokine, e.g. IL-12 to generate and expand strong antitumor immunity.

In certain embodiments, the biologically active agent comprises an epitope, e.g. a CTL or Thelper epitope for mesothelin or tyrosinase.

In certain embodiments, the biologically active agent comprises an antigen, e.g. mesothelin.

In certain embodiments, the biologically active agent comprises a ligand, e.g. the CD137 ligand to expand tumor immunity.

In certain embodiments, the biologically active agent comprises a receptor, e.g. HER-2.

In certain embodiments, the biologically active agent comprises a viral vector, e.g. an adenovirus vector encoding the E6/E7 epitopes of HPV 16 or 18.

In certain embodiments, the biologically active agent comprises a virus, e.g. HPV16 to induce an immune response to protect against cervical carcinoma or a bacterium, e.g. Heliobacter, to induce an immune response to protect against stomach cancer.

In certain embodiments, the biologically active agent is an agent capable of targeting antigens and other glycoproteins found on the surface of tumor cells. The agent can include, but is not limited to, an antibody (e.g., a monoclonal antibody (mAb), either human, humanized or chimeric), a nucleic acid (e.g., an siRNA), an aptamer, and the like. Examples of suitable targets include, but are not limited to, tumor-associated antigens (TAAs), including CD20, CD22, CD25, CD33, CD40 and CD52;tyrosine kinases, e.g., HER2/ErbB-2, EGFR, VEGFR; cell adhesion molecules, e.g., mucin 1 (MUC1), carcinoembryonic antigen (CEA1), various integrins (e.g., aVb3, a molecule enriched on vascular endothelial cells) and EpCA.

The supports used in the compositions herein are mesoporous. The term “mesoporous” as used herein means that the referenced material contains pores having average diameters between about 2 nm and about 50 nm. In certain embodiments, the pores have an average diameter between about 2 nm and about 40 nm; or between about 2 nm and about 30 nm; or about 2 nm and about 20 nm; or about 2 nm and about 10 nm. In other embodiments, the pores have an average diameter between about 5 nm and about 50 nm; or about 10 nm and about 50 nm; or about 15 nm and 50 nm; or about 20 nm and about 50 nm; or about 25 nm and about 50 nm; or about 30 nm and about 50 nm; or about 35 nm and about 50 nm; or about 40 nm and about 50 nm.

The pore size of the mesoporous support can be selected based on the type of biologically active agent which is incorporated therein. For example, the pore size can be chosen according to the following table:

| Pore Size | Biological Agent |
|---|---|
| 2 nm-5 nm | Small molecule therapeutic agents, such as the |
| | IDO inhibitor 1-methyl-tryptophan (1-MT) |
| 10 nm-20 nm | For smaller protein biomolecules (e.g., IL12/IL18) |
| 20 nm-40 nm | larger protein biomolecules (e.g., anti-CTLA4 and |
| | anti-GITR_mAbs, M.W. ~150 kD) |

The mesoporous support can comprise any material which is suitable for introduction into a physiological environment. For example, the support can be mesoporous silica, mesoporous aluminosilicate, mesoporous alumina, mesoporous clay, mesoporous metal oxide, or mesoporous polymer. In certain embodiments, the mesoporous support is a mesoporous silica.

The support can comprised particles having average diameters between 50 nm and 500 μm. In certain embodiments, the particles are between about 1 μm and about 50 μm; or between about 1 M and about 15 μm; or between 1 μm and about 30 μm.

Examples of suitable mesoporous silicas include those described in U.S. Pat. No. 6,326,326, which is hereby incorporated by reference in its entirety.

Such mesoporous supports can have surface area of greater than about 300 m²/g. In other embodiments, the support can have a surface area of greater than about 400 m²/g; or about 500 m²/g; or about 600 m²/g; or about 700 m²/g; or about 800 m²/g; or about 900 m²/g. In other embodiments, the mesoporous support can have surface area of between about 300 m²/g and 1000 m²/g; or between about 500 m²/g and 1000 m²/g; or between about 700 m²/g and 1000 m²/g.

In certain embodiments, the support is an open-celled mesoporous support. The term “open-celled” as used herein means that the cells (e.g., voids, pores, or pockets) are at least both-end opened, and may be interconnected in such a manner that a gas can pass from one to another. In certain other embodiments, the mesoporous support is an open-celled mesoporous silica.

The mesoporous support can have an optional surface functionalization. In one embodiment, the surface of the mesoporous support is functionalized. The term “surface” as used herein refers to any and all outer surface of the support and any inner surface of the porous portion of the support. A surface is considered to be “functionalized” when it has been treated or otherwise prepared in a manner which incorporates functional groups on the surface of the referenced material, where the incorporated functional groups are different that any functional groups as would normally be present on the surface of the referenced material in the absence of any functionalization. For example, silicas are known to those skilled in the art to have a surface comprising hydroxy groups; such hydroxy groups are not considered a surface functionalization as used herein. Rather, where, for example a silica has been treated in a manner familiar to those skilled in the art to provide functional groups other than hydroxy groups (e.g., thiol, amino, carboxy, sulfonic acid groups), then the silica has a surface functionalization.

The term “functional group” as used herein means a combination of atoms in a molecule, compound, composition or complex that tends to function as a single chemical entity and is responsible for the characteristic chemical properties and/or reactivity of that structure. Exemplary functional groups include, groups containing oxygen, groups containing nitrogen and groups containing phosphorus and/or sulfur. Examples of functional groups include, but are not limited to, (amine), —COOH (carboxyl), siloxane, —OH (hydroxyl), —SH (mercapto), —CONH₂ (amido), —S(O)₂OH (sulfonate), —S(O)OH (sulfinate), —OS(O)₂OH (sulfate), and chemical groups including the same. For example, functional groups may be present at the terminus of alkyl groups which are otherwise attached to the surface of the support.

In certain embodiments, the surface functionalization can comprise, for example, amino, carboxy, sulfonic acid, hydroxyl, or thiol functional groups that are positioned to be available for association with the biological agents therein. In certain embodiments, the surface functionalization can comprise, for example, amino, carboxy, sulfonic acid, or thiol functional groups that are positioned to be available for association with the biological agents therein.

In one embodiment, the surface functionalization can comprise amino groups that are positioned to be available for association with the biological agents therein. Accordingly, in certain embodiments, the mesoporous support is a mesoporous silica having a surface functionalization comprising amino groups. In certain other embodiments, the mesoporous support is an open-celled mesoporous silica having a surface functionalization comprising amino groups.

In another embodiment, the surface functionalization can comprise carboxy groups that are positioned to be available for association with the biological agents therein. Accordingly, in certain embodiments, the mesoporous support is a mesoporous silica having a surface functionalization comprising carboxy groups. In certain other embodiments, the mesoporous support is an open-celled mesoporous silica having a surface functionalization comprising carboxy groups.

In another embodiment, the surface functionalization can comprise sulfonic acid groups that are positioned to be available for association with the biological agents therein. Accordingly, in certain embodiments, the mesoporous support is a mesoporous silica having a surface functionalization comprising sulfonic acid groups. In certain other embodiments, the mesoporous support is an open-celled mesoporous silica having a surface functionalization comprising sulfonic acid groups.

In another embodiment, the surface functionalization can comprises thiol groups that are positioned to be available for association with the biological agents therein. Accordingly, in certain embodiments, the mesoporous support is a mesoporous silica having a surface functionalization comprising thiol groups. In certain other embodiments, the mesoporous support is an open-celled mesoporous silica having a surface functionalization comprising thiol groups.

The surface functionalization can be present covering about 0% to about 75% of the surface area of the mesoporous support. In certain embodiments, the surface functionalization can cover about 0% to about 70%; or 0% to about 65%; or 0% to about 60%; or 0% to about 55%; or 0% to about 50%; or 0% to about 45%; or 0% to about 40%; or 0% to about 35%; or 0% to about 30%; or 0% to about 25%; or 0% to about 20% of the surface area of the mesoporous support.

In other embodiments, the surface functionalization can be present covering about 2% to about 75%; or about 2% to about 70%; or 2% to about 65%; or 2% to about 60%; or 2% to about 55%; or 2% to about 50%; or 2% to about 45%; or 2% to about 40%; or 2% to about 35%; or 2% to about 30%; or 2% to about 25%; or 2% to about 20% of the surface area of the mesoporous support.

In certain embodiments, the surface functionalization can comprise, for example, amino, carboxy, sulfonic acid, or thiol functional ‘groups that are positioned to be available for association with the biological agents therein, wherein the surface functionalization is present covering about 2% to about 75%; or about 2% to about 70%; or 2% to about 65%; or 2% to about 60%; or 2% to about 55%; or 2% to about 50%; or 2% to about 45%; or 2% to about 40%; or 2% to about 35%; or 2% to about 30%; or 2% to about 25%; or 2% to about 20% of the surface area of the mesoporous support.

In one embodiment, the surface functionalization can comprise amino groups that are positioned to be available for association with the biological agents therein. Accordingly, in certain embodiments, the mesoporous support is a mesoporous silica having a surface functionalization comprising amino groups. In certain other embodiments, the mesoporous support is an open-celled mesoporous silica having a surface functionalization comprising amino groups. In each embodiment, the surface functionalization is present covering about 2% to about 75%; or about 2% to about 70%; or 2% to about 65%; or 2% to about 60%; or 2% to about 55%; or 2% to about 50%; or 2% to about 45%; or 2% to about 40%; or 2% to about 35%; or 2% to about 30%; or 2% to about 25%; or 2% to about 20% of the surface area of the mesoporous support.

In another embodiment, the surface functionalization can comprise carboxy groups that are positioned to be available for association with the biological agents therein. Accordingly, in certain embodiments, the mesoporous support is a mesoporous silica having a surface functionalization comprising carboxy groups. In certain other embodiments, the mesoporous support is an open-celled mesoporous silica having a surface functionalization comprising carboxy groups. In each embodiment, the surface functionalization is present covering about 2% to about 75%; or about 2% to about 70%; or 2% to about 65%; or 2% to about 60%; or 2% to about 55%; or 2% to about 50%; or 2% to about 45%; or 2% to about 40%; or 2% to about 35%; or 2% to about 30%; or 2% to about 25%; or 2% to about 20% of the surface area of the mesoporous support.

In another embodiment, the surface functionalization can comprise sulfonic acid groups that are positioned to be available for association with the biological agents therein. Accordingly, in certain embodiments, the mesoporous support is a mesoporous silica having a surface functionalization comprising sulfonic acid groups. In certain other embodiments, the mesoporous support is an open-celled mesoporous silica having a surface functionalization comprising sulfonic acid groups. In each embodiment, the surface functionalization is present covering about 2% to about 75%; or about 2% to about 70%; or 2% to about 65%; or 2% to about 60%; or 2% to about 55%; or 2% to about 50%; or 2% to about 45%; or 2% to about 40%; or 2% to about 35%; or 2% to about 30%; or 2% to about 25%; or 2% to about 20% of the surface area of the mesoporous support.

In another embodiment, the surface functionalization can comprises thiol groups that are positioned to be available for association with the biological agents therein. Accordingly, in certain embodiments, the mesoporous support is a mesoporous silica having a surface functionalization comprising thiol groups. In certain other embodiments, the mesoporous support is an open-celled mesoporous silica having a surface functionalization comprising thiol groups. In each embodiment, the surface functionalization is present covering about 2% to about 75%; or about 2% to about 70%; or 2% to about 65%; or 2% to about 60%; or 2% to about 55%; or 2% to about 50%; or 2% to about 45%; or 2% to about 40%; or 2% to about 35%; or 2% to about 30%; or 2% to about 25%; or 2% to about 20% of the surface area of the mesoporous support.

For example, functionalized mesoporous silicas having a variety of surface functionalization densities and functional groups can be prepared according to methods described in U.S. Pat. No. 6,326,326, which is hereby incorporated by reference in its entirety. For example, controlled condensation of functionalized alkylsiloxanes (e.g., G-(CH₂)ₙ—Si(OR)₃ where n is selected from 1-30 and R is hydrogen or C₁ alkyl, and G is a functional group as noted above).

Loading density of biomolecules in the mesoporous support can vary depending on the pore size, the pore volume, the spacer, the type and coverage of functional groups of the support, and the biomolecules' dimensional size and characteristics, as noted above

Advantageously, and unexpectedly, the compositions described herein have the ability to sequester large quantities of biologically active agents with respect to the mass of the support itself. For example, the compositions herein can be prepared wherein the mass ratio of the biologically active agent to the mesoporous support is greater than about 0.02 mg biologically active agent per mg of mesoporous support. In other embodiments, the mass ratio of the biologically active agent to the mesoporous support is greater than about 0.05 mg; or 0.10 mg; or 0.20 mg; or 0.30 mg; or 0.40 mg; or 0.50 mg; or 0.60 mg; or 0.70 mg; or 0.80 mg; or 0.90 mg; or 1.00 mg; or 1.10 mg; or 1.20 mg; or 1.30 mg; or 1.40 mg; or 1.50 mg; or 1.60 mg; or 1.70 mg; or 1.80 mg; or 1.90 mg; or 2.00 mg of the biologically active agent per mg of mesoporous support.

In other embodiments, the mass ratio of the biologically active agent to the mesoporous support is between about 0.02 mg and about 2.0 mg of the biologically active agent per mg of mesoporous support. In yet other embodiments, the mass ratio of the biologically active agent to the mesoporous support is between about 0.05 mg and about 2.0 mg; or about 0.10 mg and about 2.0 mg; or 0.20 mg and 2.0 mg; or 0.30 mg and 2.0 mg; or 0.40 mg and 2.0 mg; or 0.50 mg and 2.0 mg; or 0.60 mg and 2.0 mg; or 0.70 mg and 2.0 mg; or 0.80 mg and 2.0 mg; or 0.90 mg and 2.0 mg; or 1.0 mg and 2.0 mg; or 1.1 mg and 2.0 mg; or 1.2 mg and 2.0 mg; or 1.3 mg and 2.0 mg; or 1.4 mg and 2.0 mg; or 1.5 mg and 2.0 mg of the biologically active agent per mg of mesoporous support.

The outer surface of the mesoporous support can be further functionalized by binding an anti-tumor antibody to the surface. Attaching such antibodies to the surface can target the particles to specific cells within the tumor site as well as provide for better uptake and retention within the tumor. For example, locally or systemically delivered mesoporous silica containing therapeutic agents (such as an immunologically active protein) can be targeted to tumor cells expressing mesothelin (e.g., mesotheliomas, carcinomas of the ovary, and carcinomas of the pancreas), by binding a monoclonal antibody to mesothelin to the outer surface of the mesoporous silica.

In another example, a mesothelin (antigen) coated mesoporous support can be made immunogenic by use of mouse mesothelin (by being antigenically foreign) or antigen molecules that have been modified, e.g. by applying recombinant DNA technology) to localize an immunological response to the antigen at the site where the composition has been introduced by injection (e.g., at the site of a human ovarian carcinoma).

The compositions of the invention may be prepared such that the mesoporous support releases the biologically active agent at an in vitro rate of 0.1-50 μg/mg of the biologically active agent per elution at a pH 7.4, 10 mM phosphate/0.14 M NaC1 (PBS), or a simulated body fluid having a buffered pH of 7.4 with 50 mM trishydroxymethylaminomethane-HCl, or any physiological buffer in the pH range from 6.5-8.5.

For example, the mesoporous support releases about 0.1 to 100% of the biologically active agent over 1 day; or 2 days; or 3 days; or 4 days; or 5 days; or 6 days; or 7 days; or 14 days; or 21 days; or 30 days.

In other examples, the mesoporous support releases about 10% to 100%; or about 20% to 100%; or about 30% to 100%; or about 40% to 100% ; or about 50% to 100% ; or about 60 to 100%; or about 70% to 100% of the biologically active agent over 1 day; or 2 days; or 3 days; or 4 days; or 5 days; or 6 days; or 7 days; or 14 days; or 21 days; or 30 days. In certain embodiments the mesoporous support can release greater than about 75%; or greater than 85%; or greater than 95% of the biologically active agent over 7 days.

Combination Therapy

The preceding compositions may be used to provide more than one biologically active agent to a tumor (according to the methods described below). Two options for providing more than one biologically active agent include (1) incorporating more than one biologically active agent within a single mesoporous support; or (2) incorporating one or more additional biologically active agent within a one or more additional mesoporous supports, and combining the two supports to yield a blended composition.

Accordingly, in one embodiment of any of the preceding compositions, the composition comprises a second biologically active agent. The second biologically active agent can be contained within the pores of the mesoporous support; can be blended into the composition itself as a separate component; or can be adsorbed or attached to the outer surface of the mesoporous support according to methods familiar to those skilled in the art.

In another embodiment, the composition can further comprise a second mesoporous support having an optional surface functionalization, wherein the surface functionalization, when present, comprises functional groups capable of associating with a second biologically active agent; and a second biologically active agent, wherein at least a portion of the second biologically active agent is contained within the pores of the second mesoporous support.

This may be expanded to include 3, 4, 5, or more biologically active agents, each either incorporated within the same mesoporous support or loaded into separate mesoporous supports and combined to yield a blended composition. For example, anti-CTLA 4 antibody may be used to counteract immunosuppression, anti-CD3/anti-CD28 antibodies may be used to activate and expand tumor-reactive T lymphocytes, an inhibitor of IDO may be used, and an inhibitor of TGF β, each within separate mesoporous supports, as described above, loaded into the same support, or divided among 2 or 3 supports.

The surface functionalization and pore size of each support can be selected to associate with the selected biologically active agent (e.g., as noted above), and can be the same or different than the surface functionalization of any other mesoporous support of the composition(i.e. of the composition as described above).

For example, in one embodiment, the biologically active agent can comprise an antigen-specific vaccine (e.g an antigen that is expressed by the tumor being treated, e.g. mesothelin for treatment of mesothelioma, ovarian carcinoma or pancreatic carcinoma). In another embodiment, where two mesoporous supports are present, the biologically active agent contained within the first support is an antigen-specific vaccines; and the second biologically active agent (i.e., contained within the second support) is a non-specific vaccine

In another embodiment, the biologically active agent contained within the first mesoporous support can be a monoclonal antibody and the second biologically active agent can be a lymphokine, e.g. IL-12, IL-15 and/or IL18, a ligand, e.g. CD137 ligand, or a small molecule, e.g. a cytotoxic drug such as cyclophosphamide, so as to optimally activate and expand an anti-tumor response (e.g. by a combination of anti-CD3+anti-CD28 antibodies or IL-12), to decrease the impact of local immunosuppression (e.g. by anti-CTLA4 antibody and/or a drug such as cyclophosphamide or an inhibitor of IDO), to decrease the impact of immunological tolerance (e.g. by using a tumor antigen such as mesothelin which has been modified to be more immunogenic or is derived from a different species, e.g. from mouse for immunization of humans).

In another embodiment, multiple biologically active agents are present in the composition, including one or several antigens expressed by the tumor, one or several antibodies or antibody conjugates, lymphokines and/or small drug molecules that can activate tumor-reactive lymphoid cells, including T lymphocytes with CTL and helper activity, NK cells, dendritic cells and macrophages and antibodies/antibody conjugates, lymphokines and/or small molecules that can inactivate suppressive mechanisms, including such mechanisms mediated via regulatory T lymphocytes, CTLA4, IDO, an excess of tumor antigen.

Preparation of the Composition

To prepare the compositions described herein, the mesoporous support can be incubated in a solution of one or more biologically active agent under physiological conditions. Without being bound to any one theory of operation, the biologically active agents are spontaneously entrapped in mesoporous support via non-covalent interaction avoiding any harsh loading conditions. In an exemplary procedure, a pH 7.4, phosphate buffered saline (PBS) can be used containing an excess of biologically active agent. After incubation, the composition can be centrifuged, and the supernatant will be decanted. The biologically active agent loading density in the support can be calculated by subtracting the amount remaining in the supernatant from the total biologically active agent used for incubation. In one embodiment, a functionalized mesoporous silica, as described above can be incubated with a solution of a biologically active agent under physiological condition, such as a pH 7.4, phosphate buffered saline (PBS). After incubation, the FMS-biomolecule composites are centrifuged, and the supernatant decanted.

When the biologically active agents are incubated with the mesoporous support, they can be sequestered in the porous material via non-covalent interactions. This can also protect the biologically active agents because the pore size can be selected to be sufficiently small to eliminate any invading bacteria.

Further, the release rate of the entrapped biologically active agent from the mesoporous support can be controlled based on the functional groups and pore sizes. The entrapped biologically active agent can remain highly stable, and the compositions themselves can be stockpiled as drugs. Biologically active agents entrapped in mesoporous supports can be released in vivo under physiological conditions and can provide innovative therapies for many diseases that require protein drug release and delivery.

Methods for Treating Tumors

A major problem with current direct delivery techniques of therapeutic reagents into solid tumors has been resistance of the tissues to the influx of the biologically active molecules, backflow and diversion through the point of entry. This results in low quantities of remaining in the tumor tissue to be treated.

By using the compositions described herein according to the following methods, increased penetration and/or reduced backflow and diversion can be achieved through the point of entry, so that more material is introduced into and remains in the tumor, will offer considerable therapeutic advantage. In particular, the penetration of tumors with large biomolecules has been shown to be even more problematic. Mesoporous silica nanoparticles/microparticles can accumulate in tumors inside of cells as well as interstitial space. The use of this invention facilitates the penetration of biomolecules into regions of tumors with varying physical properties that may be resistant to agent penetration not incorporating the compositions of the invention.

Further, the present invention provides for sustained release of the biologically active agents once introduced near a tumor site. As used herein, “near a tumor” includes both into the tumor itself and suitably local to the tumor such that the desired biological response is elicited as could be determined by one skilled in the art (e.g.,as close as possible to the tumor site where an injection can be implemented). This advantageously delivers the biologically active agents directly to the target tissue as well as for provide for continuous treatment via slow local release over time.

By providing a controlled release of tumor antigen and immunoregulatory signals locally in tumors and at vaccination sites, mesoporous supports entrapping multiple biologically active agents (e.g., immunologically active proteins including antibodies) will induce a more effective tumor-destructive immune response with less side effects than currently available immunotherapeutic techniques for cancers.

Further, the retention of the therapeutic agent in the tissue, via the compositions of described herein, allows for longer contact of the diseased tissue with the therapeutic agent at higher and localized concentration. Because the therapeutic agents can be cytotoxic, or stimulate a cytotoxic response, the slow release does not adversely affect the patient to the point of limiting use of the therapy.

Finally, the leakage of therapeutic agents (i.e., the biologically active agents, herein) from tumors is well documented. Advantageously, this invention provides a method for retaining such agents at the tumor site that may have otherwise leaked from the target tissue. Although, as some of the agent leaks from the tumor site into the blood stream, such agent can contribute or replenish systemic concentrations, thereby acting as a depot.

Accordingly, in another aspect the present disclosure provide methods for treating a tumor comprising inserting at a site near a tumor or into the tumor in a patient in need of treatment a therapeutically effective amount of a composition according to the preceding discussion and any embodiment thereof.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. Examples of treating includes one or more of the following: (1) inhibiting the disease; for example, inhibiting a disease, condition or disorder in an individual who is experiencing or displaying the pathology or symptomatology of the disease, condition or disorder; and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “individual” or “patient,” used interchangeably, refers to any animal, including mammals, preferably mice, rats, other rodents, rabbits, dogs, cats, swine, cattle, sheep, horses, bird, fish, or primates, and most preferably humans.

As used here, a subject “in need thereof” refers to a subject that has the disorder or disease to be treated.

In the present methods, the composition may be inserted near the site of a tumor via a subcutaneous, intradermal, intramuscular, intraperitoneal, or intratumoral injection. In certain embodiments, the composition is provided by intratumoral injection. In other embodiments, the composition can be injected into the brain cavity or into the eye.

Further examples of routes for human administration are direct injection into tumor, injection into the tissues or cavities surrounding the tumor. In one embodiment, the injection site can be a body cavity; a cyst containing pathogenic cells; or a liver, pancreas, colon, lung, nervous, or central nervous system tissue.

Multiple types of cancer originate from organs located within the peritoneal cavity, e.g., pancreatic, liver, colorectal, and ovarian cancer. The peritoneal cavity also is a site for metastasis of cancer originating from organs outside of the peritoneal cavity during the late stage of disease, e.g., lung cancer. Within the peritoneal cavity, tumors can be found in pelvic and abdominal peritoneal surfaces, other peritoneal organs, e.g., intestinal mesenteries, bladder, omentum, diaphragm, lymph nodes and liver. Obstruction of the diaphragmatic or abdominal lymphatic drainage by tumor cells leads to decreased outflow of peritoneal fluid resulting in carcinomatosis or ascites.

However, intraperitoneal chemotherapy has the drawbacks including, administration through indwelling catheters, every 3 weeks for 6 treatments; infection associated with the prolonged use of a catheter; and abdominal pain due to the presentation of high drug concentrations in the peritoneal cavity. Further, intraperitoneal administration requires hospitalization and is associated with substantial costs. These reasons have contributed to the reluctance of the medical community to use intraperitoneal treatments in spite of its demonstrated survival benefits. The current invention overcomes these various deficiencies.

The instant methods advantageously provide for the localized and sustained administration of chemotherapeutic drugs in a minimally invasive fashion by localized injection of a composition of the invention near a tumor site. Accordingly, in another embodiment, a composition described herein can be injected into the peritoneal cavity. In other embodiments, a composition described herein can be injected into a peritoneal cavity for the treatment of pancreatic, liver, colorectal, ovarian, or lung cancer.

In other embodiments, a composition described herein can be injected into a peritoneal cavity for the treatment of pancreatic cancer. In other embodiments, a composition described herein can be injected into a peritoneal cavity for the treatment of liver cancer. In other embodiments, a composition described herein can be injected into a peritoneal cavity for the treatment of colorectal cancer. In other embodiments, a composition described herein can be injected into a peritoneal cavity for the treatment of ovarian cancer. In other embodiments, a composition described herein can be injected into a peritoneal cavity for the treatment of lung cancer.

A variety of tumors may be treated according to the instant methods. For example, suitable tumors include, but are not limited to, a melanoma, breast cancer, ovarian cancer, small cell lung cancer, colon cancer, rectal cancer, testicular cancer, prostate cancer, pancreatic cancer, gastric, brain, head and neck, oral, renal cell carcinoma, hepatocellular carcinoma , non-small cell lung cancer, retinoblastoma and other tumors of the eye, endometrial cancer, cervical cancer, tubal cancer.

In certain embodiments, the tumor to be treated is a melanoma. In certain other embodiments, the tumor to be treated is a breast cancer. In certain other embodiments, the tumor to be treated is ovarian cancer. In certain other embodiments, the tumor to be treated is lung cancer. In certain other embodiments, the tumor to be treated is colon cancer. In certain other embodiments, the tumor to be treated is prostate cancer. In certain other embodiments, the tumor to be treated is pancreatic cancer. In certain other embodiments, the tumor to be treated is gastric cancer. In certain other embodiments, the tumor to be treated is brain cancer. In certain other embodiments, the tumor to be treated is head and neck cancer. In certain other embodiments, the tumor to be treated is oral cancer. In certain other embodiments, the tumor to be treated is renal cell carcinoma. In certain other embodiments, the tumor to be treated is hepatocellular carcinoma. In certain other embodiments, the tumor to be treated is non-small cell lung cancer. In certain other embodiments, the tumor to be treated is colorectal cancer.

In one particular embodiment, an ovarian cancer tumor is treated by intraperitoneal injection of a composition described herein. Ovarian cancer is a group of tumors that originate in the ovaries, and can be divided into three major categories, which are named according to their cellular origin, (1) epithelial tumors, which start from the epithelial cells that cover the outer surface of the ovary; (2) germ cell tumors, which start from the germ cells that produce the ova (eggs); and (3) sex cord-stromal tumors, which are derived from the sex cord and stromal components of the developing gonad. About 90% of ovarian cancers are epithelial in origin. Epithelial ovarian cancer tends to spread in a loco-regional manner to involve the peritoneal cavity (abdominal cavity) and retro-peritoneal nodes (lymph nodes located in the retroperitoneum, the space between the peritoneum and the abdominal wall).

In another particular embodiment, an epithelial ovarian cancer tumor is treated by intraperitoneal injection of a composition described herein.

In another particular embodiment, a germ cell ovarian cancer tumor is treated by intraperitoneal injection of a composition described herein.

In another particular embodiment, an sex cord-stromal ovarian cancer tumor is treated by intraperitoneal injection of a composition described herein.

The current standard of care in the treatment of advanced ovarian cancer is cytoreductive (tumor bulk reduction) surgery followed by chemotherapy (first-line chemotherapy). However, achieving a cure for advanced ovarian cancer is very rare, the majority of patients do achieve a clinical complete remission after initial cytoreductive surgery and chemotherapy, which is rather uncommon in other advanced epithelial cancers. Over 50% of newly diagnosed patients with advanced epithelial ovarian cancer will achieve a clinical complete remission (no evidence of disease on physical examination, normal CA 125 level, normal radiographic studies) after platinum/taxane chemotherapy.

One could capitalize on this period of complete remission with maintenance therapy using the compositions herein, to prevent relapse. Such maintenance therapy may be provided concurrently or sequentially with prolonged chemotherapy treatments, such as cisplatin, paclitaxel, or a cisplatin-paclitaxel chemotherapy.

The frequency of injections will depend on the loading density or the biologically active agent, its release rate from the mesoporous support, the dose and dose interval, and can be readily determined by one skilled in the art. For example, for a composition which substantially releases its biologically active agent over the course of seven days, repeated injections may be necessary each seventh day until the desired results are obtained. In another example, for a composition which substantially releases its biologically active agent over the course of three days, repeated injections may be necessary each third day until the desired results are obtained. In another example, for a composition which substantially releases its biologically active agent over the course of fourteen days, repeated injections may be necessary each fourteenth day until the desired results are obtained. This may require, for example, 2, 3, 4, 5, 6, 7, 8, 9, or 10 or more separate injections of the instant compositions, and can be readily determined by a physician having ordinary skill in the art. In certain embodiments, the bioactive agent in the composition as described above can be selected based on the tumor to be treated. For example, the agent can be selected according to any one of the following:

| Bioactive Agent(s) | Tumor |
|---|---|
| Anti-CTLA4 mAb, Anti-CD3 + CD28 mAbsAnti-CD137 | Melanoma, ovarian cancer |
| mAb, Tyrosinase + GMCSF, mAb 7.16.4, IL-12, alone | |
| or together with IL-18, and/or anti-CTLA4 + anti-CD137 | |
| or CTLA4 + CD3 + CD28 mAbs | |
| Anti-Her2/neu, Tyrosinase, and/or Her2 peptide | Breast cancer |
| cisplatin, and/or taxol | peritoneal cancer, e.g., advanced |
| | ovarian and colon cancer |
| HuM195-Ac-225 (Humanized anti-CD33 mAb (M195) | AML (acute myelogenous leukemia) |
| conjugated to actinium 225) | |
| HuM195-Bi-213 (Humanized anti-CD33 mAb (M195) | AML |
| conjugated to bismuth 213) | |
| Anyara (naptumomab estafenatox; ABR-217620) (Anti- | Renal cell carcinoma, NSCLC (Non- |
| 5T4 Fab conjugated to superantigen variant SEA/E-20) | small cell lung cancer), pancreatic |
| | cancer |
| AS1409 (Humanized anti-ED-B fibronectin antibody | Renal cell carcinoma, melanoma |
| (BC1) conjugated to IL-12) | |
| Zevalin (ibritumomab tiuxetan) (Murine anti-CD20 | Diffuse large B-cell lymphoma |
| mAb conjugated to yttrium 90) | |
| BIIB015 (Humanized anti-Cripto mAb conjugated to | lung, colon, testicular and breast |
| DM4) | |
| BT-062 (Undisclosed mAb conjugated to DM4) | Multiple myeloma |
| Neuradiab (Murine anti-tenascin mAb (81C6) | Glioblastoma |
| conjugated to iodine 131) | |
| CDX-1307 (Human anti-mannose receptor mAb | Colorectal cancer, pancreatic cancer, |
| conjugated to hCG-β) | bladder cancer, ovarian cancer, breast |
| | cancer |
| CR011-vcMMAE (Human anti-GPNMB mAb | Melanoma, breast cancer |
| conjugated to monomethyl auristatin E) | |
| Trastuzumab-DM1 (R3502) (Humanized anti-HER2 | Breast cancer |
| mAb conjugated to DM1) | |
| Bexxar (tositumomab) (Murine anti-CD20 mAb | CLL, multiple myeloma, Hodgkin's |
| conjugated to iodine 131) | disease |
| IMGN242 (Humanized anti-CanAg mAb (C242) | Gastric cancer |
| conjugated to DM4) | |
| IMGN388 (Human anti-αv integrin mAb conjugated to | NSCLC, uterine cancer, breast cancer, |
| DM4) | prostate cancer, neuroendocrine cancer |
| IMGN901 (Humanized anti-CD56 mAb (N901) | Multiple myeloma, other cancers |
| conjugated to DM1) | |
| ¹³¹I-labetuzumab (Humanized anti-CEA mAb | Liver metastases of colorectal cancer |
| (labetuzumab) conjugated to iodine 131) | |
| IMMU-102 (⁹⁰Y-epratuzumab) (Humanized anti-CD22 | non-Hodgkin lymphomas (NHL) |
| mAb (epratuzumab)conjugated to yttrium 90) | |
| IMMU-107 (⁹⁰Y-clivatuzumab tetraxetan) (Humanized | Pancreatic cancer |
| anti-MUC1 mAb (clivatuzumab) conjugated to yttrium | |
| 90) | |
| MDX-1203 (Human anti-CD70 mAb conjugated to | Renal cell carcinoma, NHL |
| minor-groovebinding alkylating agent) | |
| CAT-8015 (Murine anti-CD22 Fv antibody fragment | Hairy cell leukemia, CLL (chronic |
| conjugated to Pseudomonas exotoxin PE38) | lymphocytic leukemia), NHL |
| EMD 273063 (hu14.18-IL2) (Humanized anti-GD2 | Melanoma, pediatric neuroblastoma |
| mAb (hu14.18) conjugated to IL-2) | |
| Tucotuzumab celmoleukin (EMD 273066; huKS-IL2) | Small-cell lung cancer |
| (Humanized anti-EpCAM mAb (KS) conjugated to IL-2) | |
| ¹⁸⁸Re-PTI-6D2 (Murine anti-melanin mAb (6D2) | Melanoma |
| conjugated to rhenium 188) | |
| Cotara (Chimeric Tumor Necrosis Therapy antibody | Glioblastoma |
| (chTNT-1B) (targeting histone H1/DNA complexes) | |
| conjugated to iodine 131) | |
| L19-IL2 (Human anti-ED-B fibronectin antibody (L19) | Renal cell carcinoma, melanoma, |
| conjugated to IL-2) | pancreatic cancer |
| Teleukin (F16-IL2) (Human anti-A1 tenascin-C | Breast cancer, ovarian cancer, lung |
| antibody (F16) conjugated to IL-2) | cancer |
| Tenarad (F16-¹³¹I) Human anti-A1 tenascin-C antibody | Cancer, hematologic malignancies |
| (F16) conjugated to-iodine 131) | |
| L19-¹³¹I (Human anti-ED-B fibronectin antibody (L19) | Cancer, hematologic malignancies |
| conjugated to iodine 131) | |
| L19-TNF (Human anti-ED-B fibronectin antibody (L19) | Melanoma, colorectal cancer |
| conjugated to TNF) | |
| PSMA-ADC (Human anti-PSMA mAb conjugated to | Prostate cancer |
| monomethyl auristatin E) | |
| DI-Leu16-IL2 (Anti-CD20 mAb conjugated to IL-2) | NHL |
| SAR3419 (Humanized anti-CD19 mAb conjugated to | NHL |
| DM4) | |
| SGN-35 (Chimeric anti-CD30 mAb conjugated to | Hodgkin's disease, anaplastic large cell |
| monomethyl auristatin E) | lymphoma, other hematologic cancers |
| CMC544 (Humanized anti-CD22 antibody conjugated | NHL |
| to calicheamicin) | |
| Rituximab (Rituxan/Mabthera; | Non-Hodgkin lymphoma |
| Genentech/Roche/Biogen Idec) (Chimeric IgG1) | |
| Trastuzumab (Herceptin; Genentech/Roche) | Breast cancer |
| (Humanized IgG1) | |
| Alemtuzumab (Campath/MabCampath; | Chronic lymphocytic leukemia |
| Genzyme/Bayer) (Humanized IgG1) | |
| Cetuximab (Erbitux; ImClone Systems/Bristol-Myers | Colorectal cancer |
| Squibb) (Chimeric IgG1) | |
| Bevacizumab (Avastin; Genentech) (Humanized IgG1) | Colorectal, breast and lung cancer |
| Panitumumab (Vectibix; Amgen) (Human IgG2) | Colorectal cancer |
| Ofatumumab (Arzerra; Genmab/GlaxoSmithKline) | Chronic lymphocytic leuakemia |
| (Human IgG1) | |
| Gemtuzumab ozogamicin (Mylotarg; Pfizer) | Acute myelogenous leukaemia |
| (Humanized IgG4) | |
| ⁹⁰Y-Ibritumomab tiuxetan (Zevalin; Biogen Idec) | Lymphoma |
| (Mouse) | |
| Tositumomab and ¹³¹I-tositumomab (Bexxar; | Lymphoma |
| GlaxoSmithKline) (Mouse) | |
| Dacetuzumab (SGN-40; Seattle Genetics) and CP- | Apoptosis in some tumors and |
| 870893 (Pfizer) | increased number of tumor-specific |
| | CD8⁺ T cells |
| Tremelimumab (CP-675,206; Pfizer) and ipilimumab | Tumor rejection, protection from |
| (MDX-010; Bristol-Myers Squibb/Medarex) | rechallenge; enhanced tumor-specific T |
| | cell responses |
| OX86 | Increase in antigen-specific CD8⁺ T |
| | cells at the tumor site; fewer MDSCs |
| | and T_{Reg} cells nd decreased levels of |
| | TGFβ; enhanced tumor rejection |
| CT-011 (Cure Tech) | Maintenance and expansion of tumor |
| | specific memory T cells populations |
| | and NK cell activation |
| BMS-663513 (Bristol-Myers Squibb) | Regression of established tumours, |
| | expansion and maintenance of CD8⁺ T |
| | cells |
| Daclizumab (Zenapax; Roche) | Transient depletion of |
| | CD4⁺CD25⁺FOXP3⁺ T_{Reg} cells⁴⁸; |
| | enhanced tumor regression and |
| | increased number of effector T cells |
| AVE9633 (huMy9-6-DM4) (Humanized anti-CD33 | AML |
| mAb Conjugate With DM4) | |
| BB-10901 (huN901-DM1) (Humanized anti-CD56 mAb | Recurrent or refractory lung cancer or |
| Conjugate With DM1) | other CD56+ solid tumors |
| CMC-544 (Humanized anti-CD22 mAb Conjugate With | B-cell NHL |
| Calicheamicin) | |
| Gemtuzumab ozogamicin (Humanized anti-CD33 mAb | Older patients with relapsed or |
| Conjugate With Calicheamicin) | untreated AML |
| huC242-DM4 (Humanized anti-CanAg mAb Conjugate | CanAg + solid tumors |
| With DM4) | |
| MLN2704 (Humanized anti-PSMA mAb Conjugate | Prostate cancer |
| With DM1) | |
| SGN-15 with Taxotere (Chimeric anti-Le(Y) mAb | Prostate cancer |
| Conjugate With Doxorubicin) | |
| A5CP + ZD2767P (Murine anti-CEA F(ab)2 fragment | Advanced CRC (colorectal cancer) |
| fused to CPG2 Conjugate With Prodrug ZD2767P) | |
| MFECP1 + ZD2767P (Murine anti-CEA scFv fragment | CEA-expressing tumors |
| fused to CPG2 Conjugate With Prodrug ZD2767P) | |
| BL22 (Murine anti-CD22 dsFv fragment Conjugate | Leukemia and lymphoma |
| With Truncated Pseudomonas exotoxin A) | |
| Hum-195/rGel (Humanized anti-CD33 antibody | Advanced myeloid malignancies |
| Conjugate With Recombinant gelonin) | |
| LMB-2 (Murine anti-CD25 scFv fragment Conjugate | Leukemia and lymphoma |
| With Truncated Pseudomonas exotoxin A) | |
| LMB-9 (Murine anti-Le(Y) dsFv fragment Conjugate | Advanced pancreatic, esophageal, |
| With Truncated Pseudomonas exotoxin A) | stomach cancer or CRC |
| SS1(dsFv)-PE38 (Murine anti-mesothelin dsFv fragment | Mesothelin-expressing tumors like |
| conjugate with Truncated Pseudomonas exotoxin) | mesothelioma, ovarian and pancreatic |
| | adenocarcinoma |
| EMD 273066 (Humanized anti-EpCAM mAb Conjugate | Ovarian, prostate, CRC and NSCLC |
| With IL-2) | |
| BiTE MT103 (Rabbit anti-CD19 scFv fragment | B-cell tumors |
| Conjugate With scFv fragment of a murine anti-CD3 | |
| mAb) | |
| rM28 (Murine anti-M-AP scFv fragment Conjugate | Metastatic melanoma |
| With scFV fragment of a murine anti-CD28 mAb) | |

In another embodiment, the method can utilize a composition comprising a biologically active agent that is an agent capable of targeting antigens and other glycoproteins found on the surface of tumor cells. The agent can include, but is not limited to, an antibody (e.g., a monoclonal antibody (mAb), either human, humanized or chimeric), a nucleic acid (e.g., an siRNA), an aptamer, and the like. Examples of suitable targets include, but are not limited to, angiogenesis inhibitor, single-target signal transduction inhibitors, multi-targeted inhibitor, cell cycle/apoptosis targeted agents, epigenetic modulator, immunomodulators, tumor-associated antigens (TAAs), including CD20, CD22, CD25, CD33, CD40 and CD52;tyrosine kinases, e.g., HER2/ErbB-2, EGFR, VEGFR; cell adhesion molecules, e.g,. mucin 1 (MUC1), carcinoembryonic antigen (CEA1), various integrins (e.g., aVb3, a molecule enriched on vascular endothelial cells) and EpCA. For example, the target can be selected according to any one of the following:

| Target | Tumor |
|---|---|
| CD20 | NHL |
| ErbB-2 | Metastatic breast cancer |
| CD33 | AML |
| CD52 | B-cell CLL |
| VEGF | CRC |
| EGFR | (CRC), (SCHN) |
| GD3 ganglioside | SCLC, Melanoma |
| mimic (e.g., | |
| anti-idiotypic mAbs) | |
| VEGFR-2/KDR | LC |
| CEA mimic | CRC or NSCLC |
| (e.g., anti-idiotypic | |
| mAbs) | |
| RANKL | PC, Multiple Myeloma |
| EGFR | advanced LC Metastatic esophagogastric cancer, |
| | Advanced LC |
| TRAIL-1 | NHL NSCLC |
| CD4 | T-Lymphoma |
| CD20 | FL, B-CLL |
| VEGF-A | Advanced ovarian cancer and CRC |
| aCD 25 | CLL, Skin cancer |
| CTLA-4 | Melanoma, Pancreatic cancer, PC, Lymphoma |
| ErBb-2 | Ovarian cancer, Breast cancer |
| X CD64 (FcγRI) | Ovarian cancer |
| CA 125 | Ovarian cancer |
| EpCam | CRC |
| CA-IX^{MN/G250} | Kidney cancer, ARCC |
| CD40 | CLL, NHL |
| a-mesothelin | Mesothelioma, ovarian, head and neck cancer |
| PEM | Ovarian cancer, Gastric cancer |
| CD33 | AML |
| CD25 | T cell leukemia/lymphoma, HL/NHL |

ALL, acute lymphocytic leukemia;
AML, acute myelogenous leukemia;
ARCC, advanced renal cell carcinoma;
BC, breast cancer;
CLL, chronic lymphocytic leukemia;
CRC, colorectal cancer;
GC, gastric cancer;
FL, follicular lymphoma;
NHL, non-Hodgkin's lymphoma;
NSCLC, non-small-cell lung cancer;
LC, lung cancer;
OC, ovarian cancer;
PC, prostate cancer

In other embodiments, various cancers may be treated using modulator for the following targets, e.g., agonists, antagonists, partial agonists, or partial antagonists of: Abl; AKT and ribosomal protein S6 kinase-1TK protein kinase;AKT protein kinase; Alk-1 protein kinase; Alpha-V chain of human integrins inhibitor (hMAb); Angiogenesis; Angiopoietin ligand-2; Apolipoprotein A (ApoA) kringle V; apoptosis protein (IAP); Apoptosis stimulator (immunoglobulin); ATPase and Hsp 90; Aurora protein kinase 1 and 2 TKI; Blocks cell division at S and G2/M; c- Met; Cadherin-3; Casein kinase II; Caspase stimulator and vascular damaging agent; CD30; CD40; CD49b; CD70; CDK-1; CDK-2; CDK4, CDK9; CDw137; Collagen I, Collagen II, Collagen III, Collagen IV and Collagen V; CXCR4 chemokine; DNA

Methyltransferase; E2F transcription factor; EGFR; EIF protein kinase; Endoglin; EpCAM; ErbB2 (e.g., ErbB2, ErbB3, ErbB4 and VEGFR-2); Erk; FGFR; Flt3; Focal adhesion kinase (Fak); G2 cell-cycle; HDAC; Hsp27; Hydroxamic acid-based HDAC; Hypoxia inducible factor-1-alpha gene; IGFRI; IgG1 chimera targeting a cell surface glycotope; IL-7; Integrin immunotoxin; Jak2; Kinesin-like protein KIF11; Kit; MEK-1 and MEK-2 protein kinase; Monocyte chemotactic protein 1 ligand; Nuclear factor kappa B; p38 MAP kinase; PDGF; PDGFR; PI3-Kinase; Pololike kinase 1; Raf 1 protein kinase; Ras GTPase; Ret; Hepatocyte growth factor receptor (HGFR); Ribosome; S100A4 receptor; SDF-1 receptor; Sphingosine-1-phosphate; Src; Tek; Telomerase; Thrombospondin-1; TKI Ron; TNF; TNF alpha; TNF superfamily receptor 12A; TRAIL-2 receptor; TrkA; uPA; VEGF; VEGFR; VEGFR1; VEGFR2; VEGFR3; MET TKI; and VGFR1.

In other embodiments, the method for treating tumors may use a composition comprising a modulator for the following targets, e.g., agonists, antagonists, partial agonists, or partial antagonists of:

| Target | Tumor |
|---|---|
| Abl & Src TKI | CML, ALL; Breast cancer, CRC, Hematological |
| | malignancies |
| Abl and Lyn TKI | AML, CML |
| Abl family and Src TKI | Hematological malignancies |
| Abl TKI | CML |
| Abl, FGFR1 and Flt-3 TKI | Hematological malignancies |
| Abl, FGFR1, Ret, TrkA and Aurora | CML |
| protein kinase TKI | |
| Abl, Jak2 and Aurora TKI | Hematological malignancies |
| Abl, Jak2, Flt-3 and AKT TKI and STAT- | ALL, AML |
| 5 stimulator | |
| AIF1 translocator | Breast & Ovarian cancer |
| AKT gene inhibitor | RCC |
| AKT protein kinase inhibitor | Prostate cancer |
| AKT protein kinase, Protein kinase C and | NHL; Glioma, CRC, NSCLC, CLL, Breast, |
| Glycogen synthase kinase-3 inhibitor | Ovarian, CNS and Prostate cancer |
| Alpha-particle-emitting radioisotope- | AML |
| linked CD33 modulator (hMAb) | |
| Aminopeptidase inhibitor | NHL |
| Angiogenesis inhibitor | NSCLC, NET, Melanoma, Prostate cancer |
| Anti-GD3 (cMAb and hMAb) | Melanoma |
| Antisense against DNA methyltransferase- | AML, MDS and RCC |
| 1 (DNMT-1) | |
| Antisense against p53 phosphothioate | AML, CLL, NHL |
| Antisense against R2 ribonucleotide | CRC, NSCLC, MDS, AML, RCC, Prostate |
| reductase mRNA | and Breast cancer |
| Antisense against TGF beta 2 | Glioma, Melanoma, CRC and Pancreatic |
| | cancer |
| Antisense against XIAP mRNA | AML, NSCLC, NHL, Pancreatic and Breast |
| | cancer |
| Antisense survivin protein modulator | AML, Prostate cancer |
| Apoptosis stimulator | Ovarian Cancer, MM, HCC, AML, CML, |
| | Leukemia and Lymphoma, Melanoma, NSCLC, |
| | Breast, Pancreatic and Prostate cancer |
| Apoptosis stimulator and cell adhesion | CLL, MM, CRC, Pancreatic and Prostate cancer |
| inhibitor | |
| Apoptosis stimulator and IL-6 antagonist | RCC, Melanoma |
| Bcl-2 gene inhibitor | Melanoma, CLL, MM, AML and NSCLC; CRC, |
| | NHL, Breast and Prostate cancer, HD, NSCLC, |
| | AML, and CML, |
| Bcl-2/Bcl-xL associated death promoter | SCLC, Lymphoma, CLL, MM, Prostate cancer |
| inhibitor | |
| Benzodiazepine receptor modulator and | Glioma and Pancreatic cancer |
| MAP kinase inhibitor | |
| B-lymphocyte antigen CD20 and CD30 | Hematological malignancies |
| immunotoxin (conjugated MAb) | |
| B-lymphocyte antigen CD20 inhibitor | Hematological malignancies, CLL, NHL |
| (hMAb) | |
| B-lymphocyte cell adhesion molecule | CLL, HCL, NHL |
| immunotoxin (recombinant Pseudomonas | |
| exotoxin A coupled to a CD22 hMAb) | |
| B-Raf protein kinase inhibitor | Melanoma |
| Cadherin-5 antagonist and vascular | CRC, NSCLC, Head & Neck, Prostate Thyroid, |
| damaging agent | Cervical and Ovarian Cancer |
| Carbonic anhydrase modulator, cell cycle | NSCLC, SCLC, CRC |
| inhibitor and apoptosis stimulator | |
| Carbonic anhydrase-IX modulator (cMAb) | RCC |
| CC chemokine receptor 4 (CCR4) | CTCL |
| modulator (hMAb) | |
| CD19 modulator (fully human antibody- | CLL |
| drug | |
| conjugate) | |
| CD20 modulator (hMAb) | CLL, NHL |
| CD22-specific cytotoxic immunoconjugate | NHL |
| of | |
| Calicheamicin (conjugated MAb) | |
| CD3 and CD19 modulator (bispecific | ALL, CLL, NHL |
| single-chain recombinant antibody) | |
| CD3 and CD20 modulator (multivalent | CLL |
| MAb) | |
| CD30 antagonist (hMAb) | HD and Lymphoma |
| CD33 modulator (hMAb) | Leukemia, AML, APL and MDS |
| CD37 modulator (small modular immuno- | CLL |
| pharmaceutical (SMIP) fusion protein) | |
| CD38 modulator (hMAb) | MM |
| CD4 modulator (hMAb) | CTCL |
| CD40 inhibitor (hMAb) | BCL, CLL and MM |
| CD43, ICAM-1 and CD55 modulator | Melanoma |
| CD80 receptor inhibitor (primatized MAb) | NHL |
| CDK and RNA synthesis inhibitor | CLL, AML, ALL, MM, Pancreatic and Ovarian |
| | cancer |
| CDK4 and CDK6 inhibitor | NHL, MM |
| CDK-4 inhibitor | MM |
| CDw137 inhibitor | NSCLC, Melanoma |
| Cell cycle inhibitor | Mesothelioma, Prostate and Ovarian cancer |
| Cell cycle inhibitor and apoptosis | NSCLC, Leukemia and Breast cancer, NSCLC, |
| stimulator | SCLC, and Ovarian cancer, AML |
| Chloride channel blocker | NET, Melanoma |
| Clusterin-inhibiting antisense | NSCLC, Breast and Prostate cancer |
| oligonucleotide | |
| CSF-1, PDGFR family Flt-3, Kit and | MDS, AML, HCC, NSCLC, RCC, Sarcoma |
| VEGFR family TKI | |
| DHFR and STAT3 inhibitor | Pancreatic cancer |
| Diamine acetyltransferase stimulator | HCC |
| Dickkopf-1 ligand inhibitor (Osteoblast | MM |
| and osteogenesis stimulator and Bone | |
| resorption inhibitor) | |
| EGFR & ErbB2 | NSCLC; Breast and Head & Neck cancer, |
| | Melanoma, CRC, Liver, Prostate and Ovarian |
| | cancer |
| EGFR family TKI | HD, NHL |
| EGFR inhibitor (hMAb) | Glioma and Pancreatic cancer; Head & Neck |
| | cancer, CRC and NSCLC |
| Endothelin ET-A receptor inhibitor | Prostate cancer |
| EpCAM and protein synthesis inhibitor | Bladder cancer |
| and immunotoxin (conjugated MAb and | |
| Ab fragment) | |
| EpCAM inhibitor and IL-2 agonist | NSCLC, SCLC, Glioma, CRC, Breast and |
| (conjugated MAb) | Ovarian cancer |
| EpCAM, CD3 and B-lymphocyte antigen | Gastric and Ovarian cancer |
| CD20 modulator (Trivalent MAb) | |
| ErbB family, RET family and VEGFR2 | NSCLC; CRC, HCC, Head & Neck, Glioma, |
| TKI | Breast, Ovarian and Thyroid cancer |
| ErbB1 ErbB2, VEGF TKI and AKT | GBM and CRC |
| protein kinase modulator | |
| ErbB2 and ErbB4 TKI | NSCLC |
| ErbB2 TK and CD3 modulator | Breast cancer |
| (Multivalent MAb) | |
| ErbB2 TKI | Breast cancer |
| ErbB2, VEGF & VEGFR2 TKI | NSCLC |
| Farnesyl transferase and ras inhibitor | AML, MDS; CML, Glioma, Melanoma, NHL, and |
| | Breast cancer |
| Fas receptor (CD95) modulator | MM |
| FGFR and VEGFR2 TKI | CRC; HCC, Sarcoma, Pancreatic and |
| | Gastrointestinal cancers |
| FGFR, PDGFR and VEGFR2 inhibitor | RCC, HCC and Breast cancer |
| FGFR, VEGFR and PDGFR TKI | NSCLC |
| Flt-3 and TrkA TKI | AML, Myelofibrosis |
| Flt-3, Kit, Tek, VEGFR2 and Hepatocyte | Thyroid cancer; NSCLC and Glioma |
| growth factor receptor (HGFR) TKI | |
| Folate receptor alpha modulator (hMAb) | Ovarian cancer |
| Ganglioside D3 (GD3) inhibitor (cMAb) | Melanoma |
| Glycosidase, Heparanase, FGFR & VEGF | HCC, Melanoma, MM, NSCLC and Prostate |
| inhibitor | cancer |
| GST P1-1 inhibitor | MDS |
| HDAC and CYP2D6 inhibitor | CTCL; Leukemia, Lymphoma, MM, MDS, RCC, |
| | CRC, Head & Neck, Breast and Prostate |
| | cancer |
| HDAC inhibitor | Glioma, NSCLC, CLL, HCC, Melanoma and Head |
| | and Neck cancer, MDS, AML, NHL, MM, |
| | Mesothelioma, CRC, Sarcoma, Thyroid and |
| | Ovarian cancer, CML, HD, MDS, AML, CLL, |
| | Pancreatic cancer, CTCL, PTCL, MM, RCC, |
| | Prostate cancer. RCC, Leukemia, Breast cancer |
| HDAC inhibitor and Bradykinin receptor | AML, HD, MM |
| modulator | |
| Hepatocyte growth factor inhibitor | Glioma and RCC |
| (HGFR) (hMAb) | |
| Hepatocyte growth factor receptor | Gastric cancer |
| (HGFR) TK inhibitor | |
| HER-2 (ErbB2) inhibitor (hMAb) | Breast cancer; Ovarian cancer, NSCLC |
| Herceptin conjugated to the antimitotic | Breast cancer |
| agent DM1, ErbB2 modulator, | |
| immunotoxin and tubulin inhibitor | |
| (Prodrug hMAb) | |
| HMFG1 based hMAb | Breast cancer |
| Hsp70 stimulator | Melanoma; NSCLC and Sarcoma |
| Hsp90 inhibitor | GIST; Melanoma |
| IGFR1 and ErbB2 TKI | Prostate cancer |
| IGFR1 inhibitor (hMAb) | NSCLC, CRC, Breast and Prostate cancer, |
| | Sarcoma, HCC, Head & Neck, Pancreatic and |
| | CRC, MM, NET, |
| IGFR1, Src and Abl TKI | ALL, CML, MM |
| IgG1 modulator (hMAb) | CRC and Gastric cancer |
| IL-2 agonist | Melanoma and CNS cancers |
| IL-2 and CD4 agonist | Head & Neck and Cervical cancer |
| IL-3 receptor modulator (MAb) | AML |
| IL-4 agonist Immunotoxin | Glioma, NSCLC, RCC, Melanoma, CRC, |
| | Pancreatic, Breast and Prostate cancer |
| IL-6 inhibitor (cMAb) | RCC, MM, NHL and Prostate cancer |
| Immunostimulant CD40 ligand receptor | NHL, CLL, HD and MM |
| inhibitor (hMAb) | |
| Immunosuppressant CD30 modulator | HD, NHL, CTCL and ALCL |
| (cMAb) | |
| Immunotoxin IL-2 receptor alpha subunit | NHL, CLL, and Melanoma |
| modulator (conjugated MAb) | |
| Inosine monophosphate dehydrogenase | Pancreatic cancer and hematological |
| (IMPDH) inhibitor | malignancies |
| Integrin inhibitor (cMAb) | NSCLC, RCC, Melanoma and Pancreatic cancer |
| Integrin inhibitor and CD51 modulator | Melanoma and Prostate cancer |
| Integrin receptor TKI | Glioma |
| Jak2 TKI | AML, CML, Hematological malignancies |
| Jak2, AKT, Extracellular signal related | MM, Prostate cancer |
| kinase-1 and Extracellular signal related | |
| kinase-2 TKI and STAT-1 and STAT-3 | |
| stimulator | |
| Kinesin-like protein inhibitor | AML, CML |
| Kinesin-like protein KIF11 and Cell cycle | NHL, HD |
| inhibitor | |
| Kinesin-like protein KIF11 inhibitor | AML, Bladder cancer, NSCLC, RCC, Leukemia, |
| | HCC, CRC, Melanoma, Head & Neck, Prostate, |
| | Breast, and Ovarian cancer |
| Kit TKI | GI cancers, MM |
| KIT, and VEGFR2 TKI | Pancreatic and Ovarian cancer |
| Lewis Y inhibitor (hMAb) | SCLC and Ovarian cancer |
| Lymphocyte function antigen-3 receptor | MM |
| (CD2) modulator (hMAb) | |
| MAP Kinase, VEGFR, PDGFR & Kit TKI | RCC, CRC, Breast and Gastrointestinal |
| | cancers |
| MAPK, PKC, AKT, and Jun N terminal | NSCLC, RCC, MM, Leukemia, CRC, Head |
| kinase inhibitor | & Neck, Pancreatic, Prostate, and Breast cancer |
| Mdm2 p53-binding protein inhibitor | NSCLC and Prostate cancer |
| MEK-1 and MEK-2 protein kinase | NSCLC, CRC, Melanoma and Pancreatic cancer |
| inhibitor | |
| Mesothelin inhibitor (cMAb) | Pancreatic cancer |
| MET receptor family and Hsp90 TKI | MM |
| MET receptor family TKI | NSCLC, Sarcoma and Pancreatic cancer |
| MET, Flt-3, KIT, Tek, VEGFR inhibitor | RCC, Head & Neck and Gastric cancer |
| mTOR inhibitor | RCC, NET, Carcinoid tumors, CRC, GIST and |
| | Pancreatic cancer, Glioma, HCC, NSCLC, Breast, |
| | Lymphoma, Gastric and Prostate cancer, Sarcoma, |
| | Breast and Gynecological cancers |
| Mucin 1 inhibitor (hMAb) | Pancreatic cancer |
| Multi-CDK inhibitor | ALL, CLL, NSCLC, NHL, MM, Head & Neck and |
| | Breast cancer, |
| Natural killer cell stimulator (hMAb) | AML, MM |
| Nicotinamide and angiogenesis inhibitor | CTCL, Leukemia and Melanoma |
| and apoptosis stimulator | |
| Nuclear factor kappa and Ikappa kinase | Leukemia |
| family inhibitor | |
| Nuclear factor kappa and I-kappa kinase | Melanoma and Pancreatic cancer |
| family inhibitor and Angiogenesis | |
| inhibitor | |
| Nuclear factor kappa B modulator | NSCLC |
| p38 MAP kinase inhibitor | MM |
| PDGFR family and Flt-3 TKI | RCC. AML, MDS, Glioma and Prostate cancer |
| PDGFR, KIT, VEGFR1 & VEGFR3 TKI | RCC and Breast cancer, RCC, NSCLC, |
| | Mesothelioma, NET, Cervical, Urothelial, Head & |
| | Neck, Sarcoma, Thyroid and Prostate cancer |
| Phosphoinositide 3-kinase (PI3K) inhibitor | MM |
| PKC inhibitor | BCC |
| PKG, cGMP phosphodiesterase and | CLL, RCC, Melanoma, Pancreatic and |
| angiogenesis inhibitor and apoptosis | Prostate cancer |
| stimulator | |
| Polo-like kinase 3 and Pololike kinase 1 | NHL |
| inhibitor | |
| Polo-like kinase-1 (PLK-1) Ser/Thr | SCLC, NSCLC, NHL |
| inhibitor | |
| Primatized CD23 inhibitor (cMAb) | CLL |
| Proteasome inhibitor | MM, WM |
| Protein Kinase C and Flt-3 TKI | AML, MDS |
| Protein kinase G and cGMP | CLL, Melanoma, RCC, Pancreatic and Prostate |
| phosphodiesterase inhibitor | cancer |
| Radioimmunotherapeutic CD29 modulator | RCC |
| and angiogenesis inhibitor (conjugated Ab | |
| fragment) | |
| Radioimmunotherapeutic CD45 inhibitor | MDS, AML, CML |
| (MAb) | |
| Radioimmunotherapeutic CD66e | CRC and Breast cancer |
| modulator | |
| Radioimmunotherapeutic CD74 inhibitor | MM, CLL, NHL |
| (hMAb) | |
| Radioimmunotherapeutic CEA Inhibitor | SCLC, NHL, CRC, HCC, Pancreatic Breast |
| | and Ovarian cancer |
| Radioimmunotherapeutic ferritin inhibitor | HD |
| (PAb) | |
| Radioimmunotherapeutic glutamate | Prostate cancer |
| carboxypeptidase II modulator (conjugated | |
| MAb) | |
| Radioimmunotherapeutic Tac inhibitor | NHL, ALL, CLL |
| (hMAb) | |
| Radioimmunotherapeutic tenascin | Glioma |
| inhibitor (conjugated MAb) | |
| Radiolabeled carbonic anhydrase-IX | RCC |
| modulator (cMAb) | |
| Retinoic acid receptor inhibitor and | RCC, NSCLC and HCC |
| apoptosis stimulator | |
| Several ribosomal proteins, HDAC, | CML |
| GPCR, PDK1 and PKA | |
| Somatostatin analog and TKI | RCC, Melanoma |
| Sphingosine kinase inhibitor | Ovarian cancer; Leukemia, Prostate, Breast, |
| | Cervical and Gynecological cancers |
| Superoxide dismutase inhibitor | MM, Prostate cancer and Lymphoma |
| Survivin protein inhibitor and apoptosis | NHL, Melanoma |
| stimulator | |
| Syk TKI | NHL |
| TACE, EGFR and ADAM-10 (sheddase) | Breast cancer |
| inhibitor | |
| Tek receptor TKI (peptibody - Fc | RCC, Breast, Gastrointestinal and Ovarian cancer |
| fragment linked to peptides) | |
| Thioredoxin inhibitor | Pancreatic and GI cancers |
| Thrombospondin-1 ligand, coagulation | NSCLC, RCC, Sarcoma, Lymphoma, Head & |
| promoter and angiogenesis inhibitor | Neck cancer |
| TLR-7 agonist | Hematological malignancies, Melanoma, Breast, |
| | Ovarian, Cervical and Uterine cancer |
| TNF-alpha agonist | CRC, HCC, SCLC and Mesothelioma |
| TRAIL receptor agonist | NSCLC, NHL |
| TRAIL-1 receptor agonist (hMAb) | NSCLC, MM, NHL and CRC |
| TRAIL-2 receptor agonist (hMAb) | Sarcoma, CRC, Pancreatic cancer, NSCLC, NHL, |
| | Sarcoma |
| Transmembrane glycoprotein NMB | Melanoma and Breast cancer |
| inhibitor and immunotoxin (hMAb and | |
| conjugated MAb) | |
| Tubulin binding CD33 modulator and | AML |
| immunotoxin (hMAb) | |
| Tubulin binding CD56 modulator | SCLC, AML, MM |
| (Prodrug, MAb, hMAb and conjugated | |
| MAb) | |
| uPA inhibitor | CRC, Head & Neck, Ovarian, Pancreatic and |
| | Gastric cancer |
| Vascular damaging agent targeting tumor | NSCLC and Breast cancer |
| endothelial cell surface PS (hMAb) | |
| VEGF & Raf protein kinase family TKI | RCC |
| VEGF inhibitor | Breast cancer |
| VEGF, CSF-1, & PDGF TKI | Thyroid and Pancreatic cancer, NSCLC and RCC, |
| | CRC |
| VEGF, FGFR, Flt-3, KIT, and PDGF TKI | AML, MM. Bladder cancer |
| VEGF, Kit & PDGF TKI | NSCLC; NET, Breast and Thyroid cancer |
| VEGF, PDGF protein family and Ras | CLL |
| protein inhibitor | |
| VEGF, Phospholipase A2 & C, STAT3, | MM, Leukemia, NET, CRC |
| AKT and IL-6 release inhibitor and TNF | |
| modulator | |
| VEGFR inhibitor | NSCLC, Ovarian cancer, Prostate cancer, |
| | Pancreatic cancer and CRC; AML, MDS, RCC, |
| | Melanoma, MM, Glioma, Thyroid, Gynecologic, |
| | and Urothelial cancers, Breast cancer |
| VEGFR, PDGFR & Kit TKI | GI cancers |
| VEGFR1 inhibitor (e.g., antisense mRNA) | SCLC, AML, ALL, MM, Melanoma, NHL, CRC, |
| | Prostate, Bladder and Thyroid cance |
| VEGFR1, VEGFR2 & VEGFR3 TKI | CRC, Glioma and Ovarian cancer; NSCLC, Head |
| | & Neck, Melanoma, RCC, CLL, AML, MDS, |
| | Mesothelioma, GIST, SCLC, HCC, Breast, |
| | Prostate, and CNS cancers |
| VEGFR2 & Raf protein kinase family | Melanoma |
| TKI | |
| VEGFR2 Inhibitor | Breast cancer, Prostate cancer, RCC, HCC, |
| | melanoma, NSCLC, Glioma |
| VEGFR2 TK inhibitor | CRC |

Abbreviations:
AA Anaplastic astrocytoma;
ALCL Anaplastic large cell lymphoma;
ALL Acute lymphoblastic leukemia;;
AML Acute myeloid leukemia;
AMM Angiogenic myeloid metaplasia;
APL Acute promyelocytic leukemia;
ASM Aggressive systemic mastocytosis;
BCC Basal cell carcinoma;
BCL B-cell lymphoma;
CEL Chronic eosinophilic leukemia;
CLL Chronic lymphocytic leukemia;
CML Chronic myeloid leukemia;
CMML Chronic myelomonocytic leukemia;
CRC Colorectal cancer;
CTCL Cutaneous T-cell lymphoma;
DFSP Dermatofibrosarcoma protuberans;
DLBCL Diffuse large B-cell lymphoma;
GBM Glioblastoma multiforme;
GI Gastrointestinal;
GIST Gastrointestinal stromal tumor;
GST P1-1 Glutathione S-transferase P1-1;
H&N Head & neck cancer;
HCC Hepatocellular carcinoma;
HCL Hairy cell leukemia;
HD Hodgkin disease;
HES Hypereosinophilic syndrome;
HL Hodgkin's lymphoma;
HRPC Hormone Refractory Prostate Cancer;
MCL Mantle cell lymphoma;
MDS Myelodysplastic syndrome;
MM Multiple myeloma;
NET Neuroendocrine tumor;
NHL Non-Hodgkin's lymphoma;
NSCLC Non-small cell lung cancer;
PG Pontine glioma;
PTCL Peripheral T-cell lymphoma;
RCC Renal cell carcinoma
SCCHN Squamous cell carcinoma of the head and neck;
SCLC Small cell lung carcinoma
TCL T-cell lymphoma

By implanting the biomolecule-nanomaterial/micromaterials locally using a variety of injection methods including subcutaneously (s.c.) or intradermally (i.d.), intramuscularly, intratumorally, etc., a long-lasting release of the biomolecules locally under physiological conditions will provide a more efficacious approach with less side effects than currently available therapeutic techniques for many diseases requiring biomolecular drug therapy.

A substantial amount of injected mesoporous support particles may be taken up by macrophages in tumors (and thereby lost from the ability to modify the immune response at the tumor site). However, this may be mediated by using mesoporous support particles with a size less likely to be taken up by macrophages.

Further, the ability of macrophages to take up silica particles may be utilized by working with particles which ‘activate’ tumor-localized macrophages so they become tumor-destructive, an approach successfully used in animal models e.g.: (Fidler, I. J., and Poste, G. Macrophage-mediated destruction of malignant utmor cells and new strategies for the therapy of metastatic disease. Springer Seminars in Immunopathology, 5: 161-174, 1982), or that facilitate the induction of a stronger anti-tumor immunity, and the uptake by macrophages and dendritic cells is influenced by the size of the nanoparticles (Ruiz et al: Polyethylenimine-based siRNA nanocomplexes reprogram tumor-associated dendritic cells via TLR5 to elicit therapeutic antitumor immunity. J. Clin. Investig. 119,2231-2244,2009).

A controlled long-lasting release of a therapeutic drug at the implanting sites will allow much less dose and much longer dose intervals and thereby provide higher efficacy and less side effects and low costs as well because the therapeutic agents are released over a prolonged period of time and do not reach the high values in the circulation which result from systemic administration. We expect this invention will bring a technological breakthrough against conventional systemic administration of drugs targeting many diseases including cancers. The invention will be able to create a new pharmaceutical industry for the production of novel and more efficacious tumor vaccines and other protein drugs, and pave the path towards new therapeutic treatments for cancers and other diseases.

The compositions herein may also be used as part of a combination therapy, where the composition is provided locally, as described above, and a second therapeutic agent is provided systemically or a second therapy method is applied. For example, the second therapy can involve providing the patient a cytotoxic agent (i.e., an agent that inhibits or prevents the function of cells and/or causes destruction of cells). Cytotoxic agents can include, but are not limited to, radioactive isotopes, as described above, such as, ¹³¹I, ¹²⁵I, ⁹⁰Y and ¹⁸⁶Re; a chemotherapeutic agent (any of those described above, or a DNA-damaging chemotherapeutic agents such as without limitation, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane)); and toxins such as enzymatically active toxins of bacterial, fungal, plant or animal origin or synthetic toxins, or fragments thereof.

Alternatively, or in addition to the preceding, a non-cytotoxic agent can be provided (i.e., a substance that does not inhibit or prevent the function of cells and/or does not cause destruction of cells) or a systemic vaccine, e.g. in the form of a tumor antigen or combination of tumor antigens that is given subcutaneously, intradermally, intramuscularly, intraperitoneally intratumorally, or intravenously, including tumor antigen combined with immunostimulatory or immunomodifying molecules with or without entrapment in mesoporous support particles. Non-cytotoxic agents include an agent that can be activated to be cytotoxic.

Alternatively, or in addition to the preceding, agents that promote DNA-damage may be provided in addition to the compositions herein, e.g., double stranded breaks in cellular DNA, in cancer cells. Any form of DNA-damaging agent know to those of skill in the art can be used. DNA damage can typically be produced by radiation therapy and/or chemotherapy.

Methods for the safe and effective administration of most of these therapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

In those embodiments where the composition is provided along with a second therapeutic method, radiation therapy may be used. Radiation therapy includes, without limitation, external radiation therapy and internal radiation therapy (also called brachytherapy). Energy sources for external radiation therapy include x-rays, gamma rays and particle beams; energy sources used in internal radiation include radioactive iodine (¹²⁵I or ¹³¹I), and from ⁸⁹Sr, or radioisotopes of phosphorous, palladium, cesium, iridium, phosphate, or cobalt. Methods of administering radiation therapy are well known to those of skill in the art. To increase the efficacy of radiation treatment, the mesoporous particles may be constructed which contain an agent (e.g. boron) which, following radiation, releases tumor-damaging radioactive particles, including such particles which have been taken up by tumor-infiltrating macrophages.

Herein, when two or more compositions and when a composition is used in a dual therapy with a second therapeutic agent or method, each may be administered to the patient simultaneously, sequentially, or alternatingly.

Below, we illustrate that immunoglobulin (IgG) molecules can be entrapped within functionalize mesoporous silica (FMS). These FMS-IgG compositions can be injected directly into mouse tumors and provide for the local release of IgG molecules. Further, the tests show the anti-tumor activity of a monoclonal antibody (mAb) to CTLA4 an immunoregulatory molecule released from FMS.

By implanting the biomolecule-nanomaterial/micromaterials locally using a variety of injection methods including subcutaneously (s.c.) or intradermally (i.d.), intramuscularly, intratumorally, intraperitoneally, etc., a long-lasting release of the biomolecules locally under physiological conditions will provide a more efficacious approach with less side effects than currently available therapeutic techniques for many diseases requiring biomolecular drug therapy. The idea can be suitable to a wider range of biomolecule-nanomaterial or biomolecule-micromaterial systems.

An important example is cancer therapy using antibodies. A fundamental aspect of cancer cells is that they have undergone extensive DNA changes and their genes mutate at a very high rate. “Loss variants” can be eliminated by localizing co-stimulatory molecules such as anti-CD137scFv at tumor sites for tumor destruction by a mechanism involving CD4+ Th1 lymphocytes and NK cells. As whole cell vaccines, tumor cells that have been transfected to express anti-CD137 scFv or CD83 have been shown to engage a larger part of the immunological repertoire than a vaccine that only targets one or two antigens. While systemic administration of certain monoclonal antibodies (Mabs), including Mabs or scFvs to CD137 and CD40, can induce anti-tumor activity, they often have side-effects by interfering with mechanisms normally protecting against autoimmunity.

### Pharmaceutical Formulations

The present disclosure further provides pharmaceutical compositions comprising a composition as described above, along with a pharmaceutically acceptable carrier. Pharmaceutically acceptable carriers are well known in the art and include sterile aqueous solvents such as physiologically buffered saline, and other solvents or vehicles such as glycols, glycerol, oils such as olive oil and injectable organic esters. The pharmaceutically acceptable carrier can further contain physiologically acceptable compounds that stabilize the compound, increase its solubility, or increase its absorption, such as, but not limited to, a salt; a buffer; a pH adjusting agent; a non-ionic detergent; and the like.

Preparations for injection can be prepared by dissolving, suspending, or emulsifying any of the compositions described above in an aqueous solvent, or a nonaqueous solvent, such as vegetable or other similar oils, synthetic aliphatic acid glycerides, esters of higher aliphatic acids or propylene glycol. In some embodiments, the formulation will include one or more conventional additives such as solubilizers, isotonic agents, suspending agents, emulsifying agents, stabilizers, and preservatives. Injectable formulations include, but are not limited to, formulations suitable for intraperitoneal injection, formulations suitable for intravenous injection, formulations suitable for intramuscular injection, formulations suitable for intraocular injection, formulations suitable for peritumoral or intratumoral injection, and formulations for subcutaneous injection.

In some embodiments, a composition as described above is suspended in normal saline. In some embodiments, a composition as described above is suspended in deionized water. In some embodiments, a composition as described above is suspended in a liquid solution comprising dextrose.

The compositions may be administered to a patient can be in the form of pharmaceutical compositions described above. These compositions can be sterilized by conventional sterilization techniques, or may be sterile filtered.

Aqueous solutions can be packaged for use as is, or lyophilized, the lyophilized preparation being combined with a sterile aqueous carrier prior to administration. The pH of the preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. In another embodiment, the composition of the invention can be pelletized to a size suitable for implantation at the site of a tumor. Alternatively, a wet paste comprising the composition and a carrier as described above can be prepared for implantation at the site of a tumor.

Kits

Also included are pharmaceutical kits useful, for example, in the treatment of tumors that include one or more containers containing a pharmaceutical composition comprising a therapeutically effective amount of a composition described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit. Other pharmaceutical kits include a first vial containing a composition (e.g., lyophilized) as described above and a second vial containing a pharmaceutically acceptable diluent, such as buffered saline, that is appropriate for preparing an injectable solution of the composition.

### EXAMPLES

The following examples are offered for illustrative purposes, and are not intended to limit the disclosure in any manner. Those of skill in the art will readily recognize a variety of noncritical parameters which can be changed or modified to yield essentially the same results.

### Example 1

We used surface-functionalized mesoporous silica (FMS) with large pores thereby yielding super-high protein loading. Unfunctionalized (as made) mesoporous silica (UMS), prepared by using non-ionic block copolymer surfactant as the template, had a pore size of 30 nm measured by the Barrett-Joyner-Halenda method, while the surface area was as great as 533 m²/g with an average bead size of 12-15 μm.

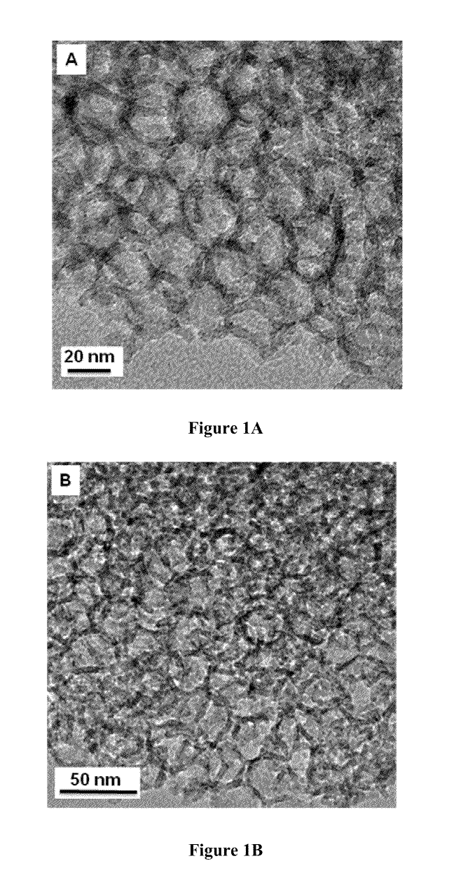

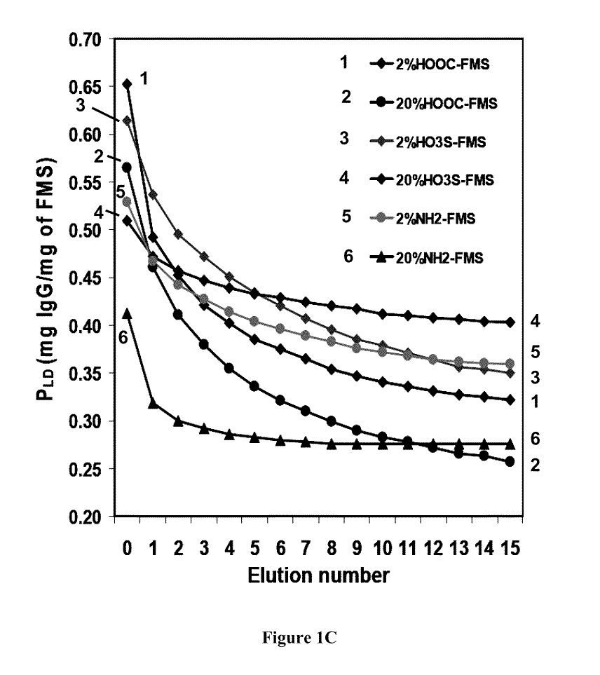

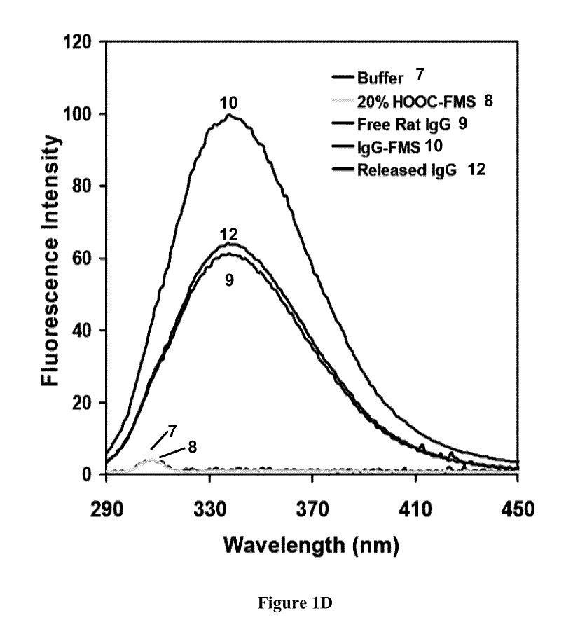

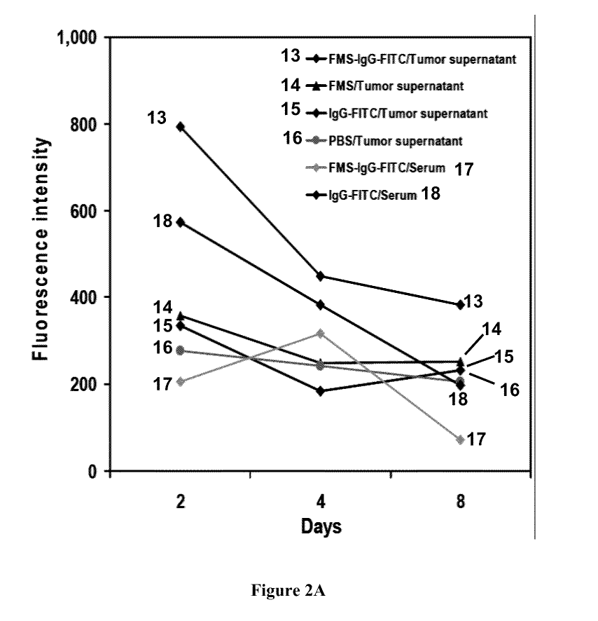

A controlled hydration and condensation reaction was used to introduce functional groups into UMS according to methods know in the art. Coverage of 2% (or 20%) HOOC-FMS or NH₂-FMS means 2% (or 20%) of the total available surface area of the mesoporous silica would be silanized with trimethoxysilane with the functional group HOOC or NH2. FIG. 1A shows the transmission electron microscopy (TEM) images of 30 nm UMS and FIG. 1B shows the corresponding 20% HOOC-FMS. There is no significant difference between the TEM images of UMS and their corresponding FMS. Unlike 3-nm and 10-nm mesoporous silica, the 30-nm mesoporous silica has a large degree of disordering, but it still reveals more or less uniform cage-like porous structure. The functional groups of HOOC, HO3S, and NH₂ would offer electrostatic, H-bond, and hydrophilic interaction with the charged amino acid residues of protein molecules.

FMS was incubated in the antibody solution, where the antibody was entrapped in FMS. We defined the protein amount (mg) of an antibody entrapped with 1 mg of FMS as the protein-loading density (PLD). We first exploited the large loading density of FMS for entrapping rat and mouse IgGs and studying their releasing ability in a physiological buffer (FIG. 1C). The resulting FMS-IgG composites were then transferred to fresh buffers and eluted multiple times to determine the release kinetics of antibody from the particles. IgGs were loaded in various FMSs including functional groups of HS, HOOC, HO₃S, and NH₂.

The results demonstrated that FMS can display remarkable loading density of rat IgGs (>0.4 mg of IgG/mg of FMS) and the subsequent controllable release of the IgG from FMS in a simulated body fluid that has ion concentrations nearly equal to those of human blood plasma and is buffered at pH 7.4 with 50 mM trishydroxymethylaminomethane and 45 mM hydrochloric acid (FIG. 1C). The similar loadings and releases were observed with mouse IgG entrapped in various FMS in pH 7.4, 10 mM sodium phosphate, 0.14 M NaCl (PBS). We found different loading densities of IgGs in various FMS, as shown in the “0 elution” data point in FIG. 1C. The protein contents of the supernatants in between each cycle of shaking-elution-centrifugation were measured. A decreasing PLD was observed along the series of elutions (FIG. 1C). The 20% HOOC-FMS and 2% HO₃S-FMS also displayed faster releasing rates than other FMSs under the identical elution solutions (FIG. 1C).

These results reflected the difference of the comprehensive interaction of IgG with various FMSs; that is, the electrostatic, H-bond, and hydrophilic interaction with the charged amino acid residues of protein molecules. FIG. 1D shows fluorescence emission spectra of the free rat IgG, the entrapped IgG in FMS, and the released IgG from FMS. Fluorescence emission was monitored at the excitation wavelength of 278 nm, allowing excitation of both tyrosinyl and tryptophanyl residues. Comparing the free IgG to FMS-IgG (FIG. 1D), there was no dramatic emission peak shift but increased emission intensity because of the interaction of IgG with FMS, which might result in less exposure of tyrosinyl and tryptophanyl residues to the aqueous environment. It is noteworthy that the released IgG displayed similar fluorescence spectra to that of the free IgG prior to the entrapment, indicating that the interaction of FMS with IgG did not induce dramatic change on the IgG protein structure.

To confirm that the released antibody can still maintain the binding activity to its antigen, anti-calf intestinal alkaline phosphatase (anti-CIP) was incubated with various FMS. Then, the antigen binding activities of the released anti-CIP from FMS over time were measured. The results have demonstrated that the released anti-CIP maintained their binding activity.

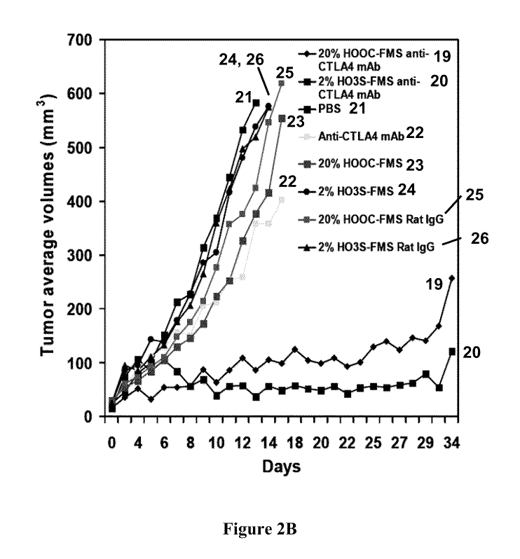

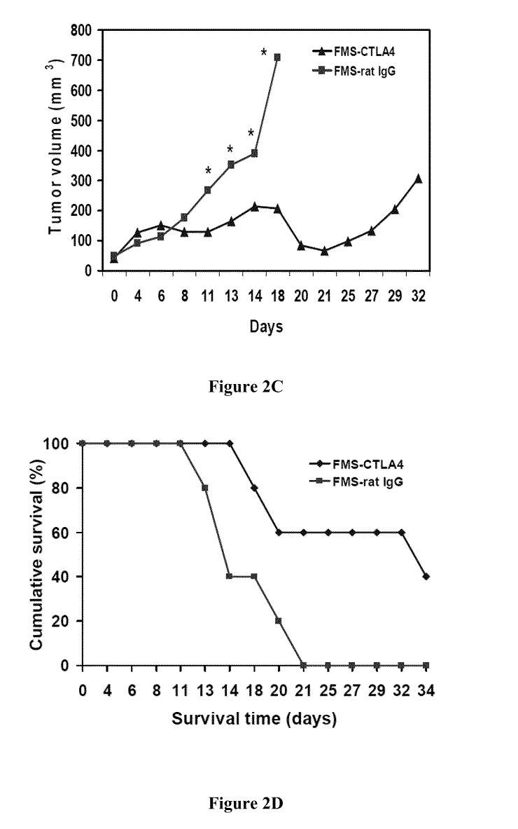

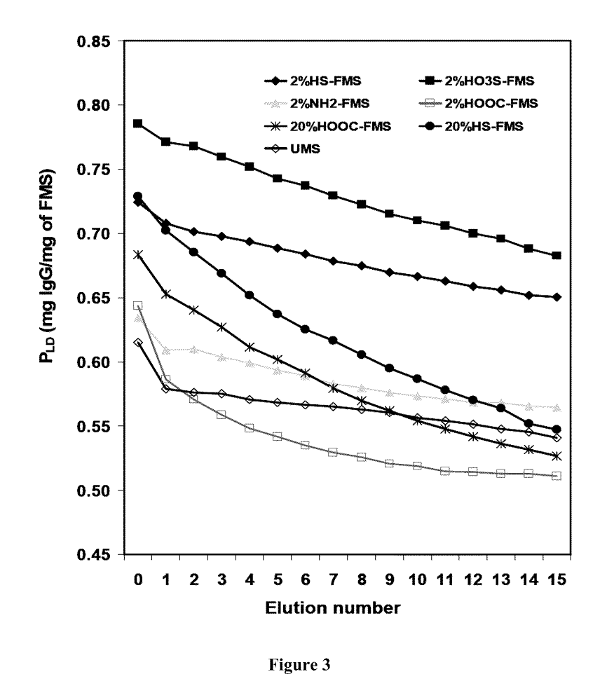

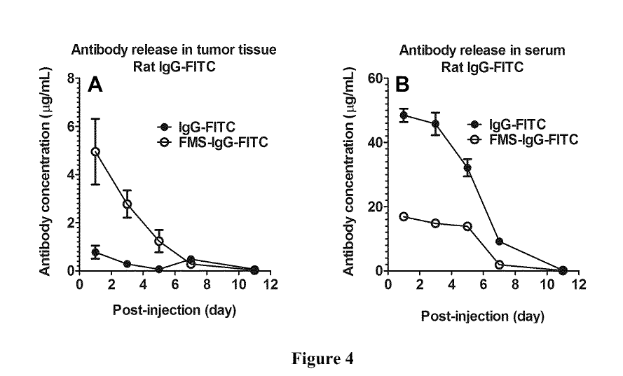

To monitor the local release of the antibodies from FMS in mice, we injected FITC-labeled-rat IgG (IgG-FITC) and FMS-IgG-FITC into established mouse melanomas derived from subcutaneous (s.c.) injection of cells from the SW1 clone of the K1735 melanoma. There were two groups of mice, in which tumors were injected with the same amount of IgG-FITC with or without entrapment in 20% HOOC-FMS particles. Tumors and sera were harvested after 2, 4, and 8 days, and tumors were digested with digestion buffer (Hank's balanced salt solution with collagenase, hyaluronidase, and DNase). The tumor lysates were cleared by centrifugation, and the supernatants were collected. The fluorescence intensity was measured in the serum and tumor supernatants. The unreleased IgG-FITCs inside FMS were not counted because that part stays with the cell pellet. At the tumor site on day 2, all initially injected IgG-FITC (no FMS) was completely gone (see the control experiments, FIG. 2A). In sharp contrast, for the FMS-IgG-FITC on day 2, and even on days 4 and 8, there was still significant free IgG-FITC released from the FMS particles at the tumor site. In the case when FMS-IgG-FITC was injected into the tumor, we got a higher FITC reading in tumor supernatant accompanied by a lower one in the serum (FIG. 2A).

The FMS particles continued releasing the IgG-FITC, otherwise we would not have detected any free IgG-FITC after 2, 4, and 8 days, because IgG-FITC that is not entrapped in FMS particles is distributed very quickly (FIG. 2A). Interestingly, the data were the opposite when IgG-FITC (no FMS) was injected into the tumor; that is, we got a lower FITC reading in tumor supernatant accompanied by a higher one in the serum. The data clearly show that, after euthanization, FMS-IgG-FITC-injected mice had more antibodies in the whole tumor cells than did the IgG-FITC mice in the absence of FMS. These results indicate that FMS entrapping with IgG prolonged the antibody stay at the tumor site and thus facilitates sustained antibody release in tumors, offering an advantage over simply injecting antibodies into tumors.

Monoclonal antibodies have been used to treat many medical conditions, including cancer. For example, a systemic administration of a mAb to the immunoregulatory molecule CTLA4 has representative results from each treatment group. The results demonstrate that FMS-anti-CTLA4 inhibited tumor growth. We saw no evidence of toxicity from injecting FMS particles into tumors. In particular, the anti-tumor activity of FMS-Anti-CTLA4 (>50% tumor regression) was much more potent than that of anti-CTL4 alone (without FMS). We have repeated the experiment and got similar results (FIGS. 2C & 2D).

We conclude that immunoglobulins can be loaded in FMS particles to provide long-lasting local release, and our data indicates that an FMS-entrapped anti-CTLA4 IgG mAb induces a better therapeutic response than the same amount given systemically. The experimental conditions, the rate and durability of the mAb release from FMS particles can be adjusted by changing the pore size and the functional groups of FMS (FIG. 1C).

A similar approach of local release can be applied to other mAbs as well as to lymphokines and other immunologically active proteins, delivered alone or in combination, and that a long-lasting local release will cause more effective tumor destruction with less dose amount, longer dose intervals, and fewer side effects than systemic administration. Entrapment into FMS particles may also be used as a tool to compare the therapeutic efficacy of various immunomodulatory proteins in the tumor microenvironment to guide the selection of the most effective molecules for tumor targeting.

### Example 2

### Relative Activity of Continuously Released Antibody from FMS

To confirm that a released antibody can still maintain the binding activity to its antigen, we incubated commercially available rabbit anti-calf intestinal alkaline phosphatase (anti-CIP) with various FMS. The binding activity for antigen of the released anti-CIP from FMS was measured by surface plasma resonance to determine whether FMS binding had any deleterious effect on antibody activity. The activity was calculated assuming that if 100% active, 148 RU of the antibody would exhibit a maximum antigen binding of 116 RU, 116/148=88% active and assigned a relative activity ratio of 1. Thus, the relative activities of the released anti-CIP from FMS were measured (Table 1). Although there is some data variation, the released anti-CIP maintained their binding activity.

TABLE 1
Relative activity of continuously released antibody from FMS*

Relative binding
activity of anti-CIP
released from FMSs

| FMSs | 24 h | 48 h | 72 h | 96 h |
|---|---|---|---|---|
| 20% HO₃S-FMS | 0.76 | 1.26 | 1.14 | 1.14 |
| 20% HOOC-FMS | 1.25 | 0.77 | 1.15 | 1.02 |
| 20% HS-FMS | 1.18 | 1.32 | | |
| 20% NH₂-FMS | 0.82 | 0.94 | 1.09 | 1.10 |
| 2% HO₃S-FMS | 0.93 | 1.00 | 0.78 | 1.18 |

*Sample preparation: Anti-CIP was shaken with individual FMS in pH 7.4, PBS for every 24 h, then centrifuged and the supernatant was taken out and measured. The same volume of the fresh buffer was added after taking the supernatant out each time.

FMS and FMS-antibody. Hexagonally ordered mesoporous silica (SBA-15) of pore size 300 Å and surface area of 533 m²/g were prepared according to procedures modified from our earlier work. In a typical preparation of mesoporous silica with 300 Å pores, 12.0 g of Pluronic P-123 (MW=5,800) was dissolved in 2 M HCl solution (360 mL) at 40° C. Then 18.0 g of mesitylene and 25.5 g of tetraethylorthosilicate (TEOS) were added to the milky solution and stirred for 18 h at the same temperature. The mixture was transferred into a Teflon-lined autoclave and heated up to 100° C. for 24 h without stirring. The white precipitate was collected by filtration, dried in air, and finally calcined at 550° C. for 6 hours. A controlled hydration and condensation reaction was used to introduce functional groups into unfunctionalized mesoporous silica (UMS). A coverage of 2% (or 20%) HOOC-FMS, HO₃S- or NH₂-FMS means 2% (or 20%) of the total available surface area of the mesoporous silica would be silanized with the trimethoxysilane with the functional group HOOC—, HO₃S—, or NH₂—. In a typical procedure of 2% HOOC-FMS synthesis (300 Å pores), 1.0 g of mesoporous silica was first suspended in toluene (60 mL) and pretreated with water (0.32 mL) in a three-necked 250 mL round-bottom flask, which was fitted with a stopper and reflux condenser. This suspension was stirred vigorously for 2 h to distribute the water throughout the mesoporous matrix, during which time it became thick and homogeneous slurry. At this point, 15.5 mg of tris-(methoxy)cyanoethylsilane (TMCES, MW=175.26) was added and the mixture was refluxed for 6 h. The mixture was allowed to cool to room temperature and the product was collected by vacuum filtration. The treated mesoporous silica was washed with ethyl alcohol repeatedly and dried under vacuum. To hydrolyze cyano groups (CN— would be hydrolyzed into HOOC— as the functional group), 10 mL of 50% of H₂SO₄ solution was added to the mixture and refluxed for 3 h. The product was filtered off and washed with water extensively. Other samples were synthesized by the same procedure except different amounts of organosilanes were added based on their surface areas, and no hydrolysis step when functionalizing with tris- (methoxy)aminopropylsilane (TMAPS, NH₂— as the functional group) and tris-(methoxy)mercaptopropylsilane (TMMPS, HS— as the functional group). HO₃S-FMS was prepared via oxidation of HS-FMS by 30% (w/w) H₂O₂. Typically, an aliquot of 2.0-8.0 mg of FMS was added in a 1.8-mL tube for incubation with 200-1600 μL of the antibody stock. Based on the preliminary experiments, at least 0.5-1.0 mg antibody was used for incubation with per mg of FMS so that FMS was loaded to saturation with the antibody. The incubation was carried out at 18-21° C. shaking at 1400 min⁻¹ on an Eppendorf Thermomixer 5436 for 12-24 h. The antibody stock in the absence of FMS was also shaken under the same conditions for comparison. Then the FMS-antibody composites were separated by centrifugation. The amounts of proteins were measured by Bradford method using bovine γ globulin as standards.

High resolution TEM was carried out on a Jeol JEM 2010 Microscope with a specified point-to-point resolution of 0.194 nm. The operating voltage on the microscope was 200 keV.

Mice and tumor cells. Six- to eight-week-old female C3H/HeN mice were purchased (Charles River Laboratories, Wilmington, Mass.). The SW1C clone of the K1735 melanoma is of C3H/HeN origin. 3 The animal facilities are ALAC certified, and our protocols are approved by Univerity of Washington's IACUC Committee.

In vivo antibody release assay. 6-8 week female C3H mice were transplanted s.c. on one side of the back with 106 SW1-WT tumor cells. When the tumor size reached 3 mm by 3 mm, 0.885 mg of 20% HOOC-FMS Rat IgG-FITC, containing 0.1 mg Rat IgG-FITC, was injected into the tumor. Mice were euthanized at the indicated time point. The tumors were removed, cut into small pieces, digested in the tumor digestion medium (Hank's balanced salt solution with collagenase, hyaluronidase, and DNase) for 2 h at 37° C. with shaking. The supernatant was harvested by centrifuge. The fluorescence intensity was measured at OD535 by ELISA reader.

Animal studies. Mice were transplanted s.c. on both sides of the back, with 106 tumor cells. When the tumors were 3-5 mm in mean diameter, mice in the experimental groups were injected s.c. with 1.8 mg FMS particle entrapping 0.5-0.8 mg anti-CTLA4,4 or control antibody (rat IgG), while the control groups got PBS or anti-CTLA4 by i.p. Tumor growth was assessed by measuring the two largest perpendicular diameters and reported as average tumor volume (in mm³) by the formula (length2×width/4). Statistical analysis of these results was done by t-test and one-way ANOVA test. All statistical tests were two-sided.

### Example 3

### In Vivo Release of Antibodies from FMS

To monitor the local release of the antibodies from 20% HOOC-FMS in mice, we intratumorally injected one dose of 0.1 mg IgG-FITC and FMS entrapped with 0.1 mg IgG-FITC into established mouse melanomas derived from subcutaneous (s.c.) injection of cells from the SW1 clone of the K 1735 melanoma. The concentration of IgG-FITC in the serum and the tumor supernatant were measured using fluorescence reader (FIG. 4). The in vivo preliminary data shows that the free IgG-FITC injected i.t. without FMS disappeared rapidly, but in contrast, there was a significant instant release of IgG-FITC from the FMS particles at the tumor site monitored over days, indicating that the FMS-IgG composite prolonged the antibody stay at the tumor site and the antibody was continuously and gradually released from FMS at the tumor site over days (FIG. 4). Multiple factors of FMS, distinctness of IA biomolecules and the dose amount will affect the drug release kinetics.

### Example 4

### Decreased Toxicity from Local Release of Antibodies from FMS

Injection of the same amount of anti-CD3+anti-CD28 monoclonal antibody was less toxic to tumor-bearing mice when entrapped in FMS particles than when injected without such entrapment, and the FMS approach may, therefore, make it possible to clinically use this antibody combination, which can effectively activate and expand tumor-reactive T lymphocytes (Hellstrom, I., Ledbetter, J. A., Scholler, N., Yang, Y., Ye, Z., Goodman, G., Pullman, J., Hayden-Ledbetter, M., and Hellstrom, K. E. CD3-mediated activation of tumor-reactive lymphocytes from patients with advanced cancer. Proc Natl Acad Sci USA, 98: 6783-6788, 2001), but has too high toxicity to be used without entrapment in FMS particles.

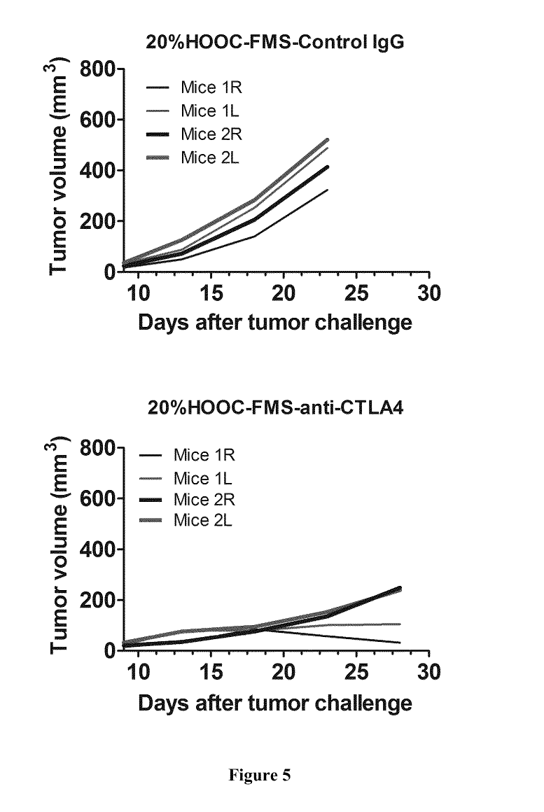

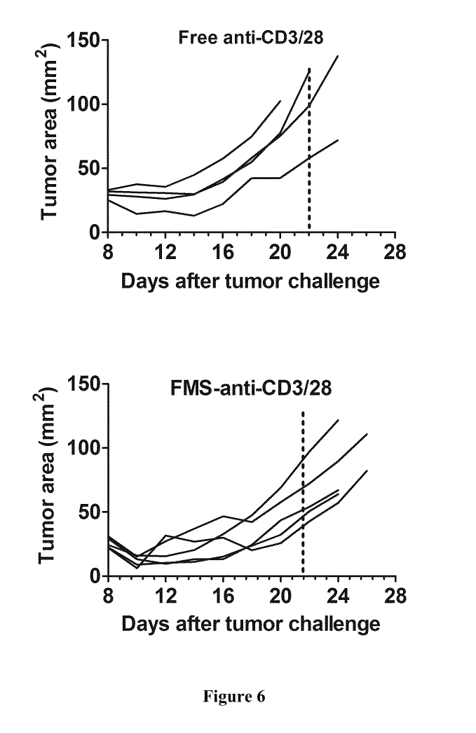

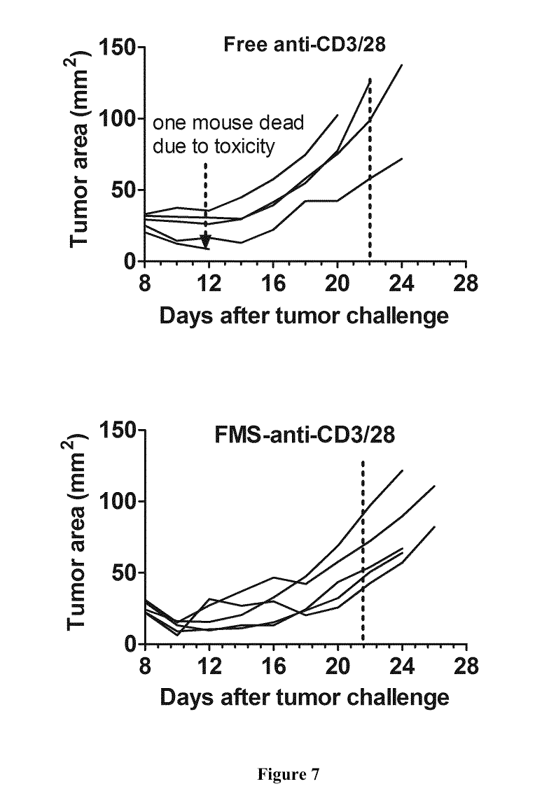

FIG. 5 shows regression also of untreated tumors in mice similar to those in FIG. 2C but carrying two established SW1 melanomas, one of which was treated by injection of FMS particles containing anti-CTLA4 Mab while the other tumor was left untreated. FIG. 6 shows anti-tumor activity on established SW1 melanoma of anti-CD3+anti-CD28 monoclonal antibody entrapped in FMS particles but not of anti-CD3+anti-CD28 antibody. FIG. 7 shows an experiment similar to that in FIG. 6 but with a double antibody dose (1200 μg/mouse) where one mouse in the ‘free’ antibody group died from toxicity 4 days after onset of treatment.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby. Each referenced document herein is incorporated by reference in its entirety for all purposes. Changes can be made in the composition, operation and arrangement of the method of the present invention described herein without departing from the concept and scope of the invention as defined in the following claims.

We claim:

1. A method for treating a tumor comprising inserting at a site near a tumor in a patient in need of treatment a therapeutically effective amount of a composition comprising
(i) a mesoporous support having an optional surface functionalization, wherein the surface functionalization, when present, comprises functional groups capable of associating with the biologically active agent; and
(ii) at least one biologically active agent, wherein at least a portion of each biologically active agent is contained within the pores of the mesoporous support.

2. The method of claim 1, wherein the mass ratio of the biologically active agent to the mesoporous support is greater than about 0.02 mg biologically active agent per mg of mesoporous support.

3. The method of claim 1, wherein the support is an open-celled mesoporous support.

4. The method of claim 1, wherein the biologically active agent comprises a pharmaceutical, a protein, an antibody, a nucleic acid, or a mixture thereof

5. The method of claim 4, wherein the biologically active agent comprises an antibody.

6. The method of claim 1, wherein the biologically active agent comprises a vaccine.

7. The method of claim 1, wherein the biologically active molecule is an antibody-conjugate.

8. The method of claim 1, wherein the mesoporous support selected from the group consisting of a mesoporous silica, aluminosilicate, mesoporous alumina, mesoporous clay, mesoporous metal oxide, mesoporous metal hydroxide, and mesoporous polymer.

9. The method of claim 8, wherein the mesoporous support is a mesoporous silica.

10. The method of claim 1, wherein the surface functionalization comprises amino, carboxy, sulfonic acid, or thiol functional groups.

11. The method of claim 10, wherein about 0% to about 75% of the surface area of the mesoporous support comprises the surface functionalization comprising amino, carboxy, sulfonic acid, hydroxyl, or thiol functional groups.

12. The method of claim 1, wherein the composition further comprises a second biologically active agent.

13. The method of claim 1, wherein the composition further comprises one or more additional mesoporous supports, each having an optional surface functionalization, wherein the surface functionalization, when present, comprises functional groups capable of associating with one or more biologically active agents; and one or more additional biologically active agents, wherein at least a portion of each additional biologically active agent is contained within the pores of the mesoporous supports.

14. The method of claim 1, wherein the injection site is
a. a peritoneal cavity;
b. a cyst containing pathogenic cells;
c. or a liver, pancreas, colon, lung, nervous, or central nervous system tissue.

15. The method of claim 1, wherein the inserting selected from the group consisting of a subcutaneous, intradermal, intramuscular, intraperitoneal, and intratumoral injection.

16. The method of claim 1, wherein the tumor is selected from the group consisting of a melanoma, breast cancer, ovarian cancer, small cell lung cancer, colon cancer, rectal cancer, testicular cancer, prostate cancer, pancreatic cancer, gastric, brain, head and neck, oral, renal cell carcinoma, hepatocellular carcinoma , non-small cell lung cancer, retinoblastoma, eye tumors, endometrial cancer, cervical cancer, and tubal cancer.

17. The method of claim 1, wherein the inserting is an intraperitoneal injection and the tumor is ovarian cancer.

18. The method of claim 1, wherein the inserting is an intratumoral injection.

19. A composition comprising (i) a mesoporous support having an optional surface functionalization, wherein the surface functionalization, when present, comprises functional groups capable of associating with a biologically active agent; and (ii) at least one biologically active agent, wherein at least a portion of each biologically active agent is contained within the pores of the mesoporous support.

20. A pharmaceutical composition comprising the composition of claim 19 and a pharmaceutically acceptable carrier.
